(19) United States
(12) Patent Application Publication
(10) Pub. No.: US 2022/0192345 A1
(43) Pub. Date: Jun. 23, 2022

(54) SURFACE ENERGY ENHANCING FLUID AND APPLICATIONS ON LIVING TISSUES

(71) Applicant: Booker Holdings, LLC, Newport Beach, CA (US)

(72) Inventor: Charles Kent Booker, Newport Beach, CA (US)

(21) Appl. No.: 17/692,749

(22) Filed: Mar. 11, 2022

Related U.S. Application Data
(63) Continuation of application No. 17/025,825, filed on Sep. 18, 2020.
Application No. 17/025,825 is a continuation of application No. PCT/US2019/038562, filed on Jun. 21, 2019.
(60) Provisional application No. 62/689,657, filed on Jun. 25, 2018.

(51) Int. Cl. A45D 44/00; A61M 35/00; A61K 8/22; A61K 8/49; B01D 53/04; H01T 19/00

(52) U.S. Cl. CPC A45D 44/002; A61M 35/30; A61K 8/22; A61K 8/4973; B01D 53/04; B01D 2257/106; A61M 2202/0216; A61M 2205/75; A61M 2210/0606; A61M 2210/08; H01T 19/00

(57) ABSTRACT

Devices and methods can be used to apply an adhesion promoter, for example, ozone, over the surface of the biological tissues, before and/or concurrently with the application of the cosmetics to improve the durability and/or appearance of the cosmetics on the biological tissues. The devices can include a recirculation system and/or a filter system to reduce the likelihood of the chemical being released into the atmosphere and/or to increase oxidation of the chemical generated by the device after use of the device.

### INCORPORATION BY REFERENCE

This application is a continuation of U.S. patent application Ser. No. 17/025,825, filed Sep. 18, 2020, which is a continuation of International Patent Application No. PCT/US2019/038562, filed Jun. 21, 2019, which claims the priority benefit of U.S. Provisional Application No. 62/689,657, filed Jun. 25, 2018, the entirety of which is incorporated herein by reference for all purposes.

### FIELD

The present disclosure relates to methods and systems for applying a chemical to surfaces of living tissues.

### BACKGROUND

Most cosmetics involve applying a coating or dye to skin, fingernails, and hair. Cosmetics have a limited useful life once applied, for example, over the course of about one day for make-up such as foundation and lipstick. Hair dyes, spray tans, nail polish and other applied cosmetics also may last a matter of days or weeks. Customers become frustrated when these cosmetics do not perform in a robust way, such as the nail polish flaking or chipping after a short period of time (such as within about 2-3 days), the hair dye or spray tanning fading too quickly (such as within about 3-5 days), or the face foundation, mascara, and/or lipstick clumping or crumbling away before the customer wants it to (such as within a few hours of application).

### SUMMARY

A device disclosed herein can be used to increase surface energy of a portion of a digit. The device may include a gas generator configured to generate a surface energy enhancing fluid that can be in fluid communication with the portion of the digit.

A device disclosed herein for increasing surface energy of body tissues may also include a gas generator configured to generate a surface energy enhancing fluid and a tissue-interfacing attachment. The surface energy enhancing fluid may be configured to increase the surface energy of the body tissues.

A method disclosed herein for increasing surface energy of a portion of a body may comprise delivering a surface energy enhancing fluid to the portion of the body from a delivery device and increasing the surface energy of the portion of the body by 10 mN/m to 20 mN/m.

A method disclosed herein for increasing surface energy of a portion of a body comprises delivering a surface energy enhancing fluid to the portion of the body; and stopping delivering the surface energy enhancing fluid after a duration.

A method disclosed herein for increasing surface energy of a portion of a body may comprise generating a surface energy enhancing fluid using a gas generator; delivering the surface energy enhancing fluid to a surface of the portion of the body placed in a substantially sealed chamber; and terminating the generating and/or delivering of the surface energy enhancing fluid after a duration.

A method disclosed herein for improving cosmetics application may comprise increasing a surface energy of a body part; and applying cosmetics to a surface of the body part within 3 minutes after increasing a surface energy of a body part.

A method disclosed herein for improving cosmetics application may comprise placing a body part of a user in a substantially sealed chamber, wherein a surface energy enhancing fluid may be delivered to a surface of the body part; removing the body part from the chamber; and applying cosmetics to the surface of the body part within a duration of removing the body part from the chamber.

A modular cosmetics applicator disclosed herein may be used to increase surface energy of body tissue. The modular cosmetics applicator may comprise an inlet configured to be in fluid communication with a gas generator configured to generate a surface energy enhancing fluid, and a lumen in fluid communication with the inlet and a tissue interface portion.

A cosmetic appliance disclosed herein may be configured to increase surface energy of body tissue before application of cosmetics to the body tissue. The appliance may comprise a gas generator configured to generate a surface energy enhancing fluid, and a lumen in fluid communication with the gas generator and a cosmetics applicator. The surface energy enhancing fluid generated by the gas generator may be configured to flow through the lumen to the cosmetics applicator.

A device disclosed herein may be configured to temporarily increase surface energy of body tissues. The device may comprise a substantially sealed pathway for the surface energy enhancing fluid during use of the device on the body tissues sealed to inhibit the surface energy enhancing fluid from exiting the device. The substantially sealed pathway may include a gas generator configured to generate a surface energy enhancing fluid, a gas chamber configured to provide exposure of the body tissues to the generated surface energy enhancing fluid, a first conduit configured to deliver the generated surface energy enhancing fluid from the gas generator to the gas chamber, and a second conduit configured to recirculate remaining surface energy enhancing fluid from the gas chamber to the gas generator.

In some examples, a device for increasing surface energy of a portion of a digit comprises a gas generator configured to generate a surface energy enhancing fluid, and a housing comprising an opening for insertion of the portion of the digit into fluid communication with the surface energy enhancing fluid.

The portion digit may comprise a finger including a fingernail. The portion digit may comprise a toe including a toenail. The housing may comprise a plurality of openings including the opening. The plurality of openings may comprise five openings. One opening of the five openings may be offset from four openings of the five openings. The device may further comprise a filter configured to absorb the surface energy enhancing fluid when the device is not in use. The filter may comprise activated charcoal. The device may further comprise a controller configured to deactivate the gas generator after a duration. The duration may be between 15 seconds and 35 seconds. The duration may be 30 seconds. The duration may be configured to increase the surface energy of the portion of the digit by 10 mN/m to 20 mN/m. The controller may be configured to emit a signal after a second duration after the duration. The second duration may be between 1 min and 4 min. The second duration may be 3 min. The signal may comprise an audible alarm. The signal may comprise a visual alarm. The gas generator may be configured to generate the surface energy enhancing fluid between 0.2 g/hour and 0.8 g/hour. The gas generator may be configured to generate the surface energy enhancing fluid between 0.6 g/hour. The surface energy enhancing fluid may comprise ozone. The gas generator may be configured to generate the ozone by applying an electrical current to oxygen. The oxygen may come from air. The device may further comprise a recirculation conduit, wherein a concentration of the surface energy enhancing fluid may increase after every circulation of the surface energy enhancing fluid through the recirculation conduit. The device may further comprise a blower configured to pressurize the surface energy enhancing fluid. The device may further comprise a sealing membrane configured to substantially seal the housing upon insertion of the portion of the digit. The sealing membrane may be configured to inhibit the surface energy enhancing fluid from exiting the housing.

In some examples, a device for increasing surface energy of body tissues comprises a gas generator configured to generate a surface energy enhancing fluid, a housing enclosing the gas generator and the blower, and a tissue-interfacing attachment coupled to the housing. The surface energy enhancing fluid may be configured to increase the surface energy of the body tissues.

The device may further comprise a blower configured to pressurize the surface energy enhancing fluid. The device may further comprise a delivery conduit between the housing and the tissue-interfacing attachment, wherein the delivery conduit may be configured to deliver the surface energy enhancing fluid via the tissue-interfacing attachment to the body tissues. The device may further comprise a recirculation conduit attached between the housing and the tissue-interfacing attachment, wherein the recirculation conduit may be configured to return the surface energy enhancing fluid from the tissue-interfacing attachment to the housing. A concentration of the surface energy enhancing fluid may increase after every circulation of the surface energy enhancing fluid through the recirculation conduit. When the tissue-interfacing attachment is applied to a surface of the body tissues, the device may be substantially sealed to inhibit the surface energy enhancing fluid from exiting the device. The device may further comprise a filter configured to absorb the surface energy enhancing fluid when the device is not in use. The filter may comprise activated charcoal. The device may further comprise a controller configured to deactivate the gas generator after a duration. The duration may be 10 seconds to 35 seconds. The gas generator may be configured to generate the surface energy enhancing fluid at 0.6 g/hour. The surface energy enhancing fluid may comprise ozone. Ozone may be generated by applying an electrical current to oxygen in the atmospheric air. The tissue-interfacing attachment may comprise a face mask. The face mask may comprise a sealing membrane extending around a perimeter of the face mask. The face mask may comprise an opening configured to let a mouth of a user breathe through the opening when the device is in use. The face mask may comprise a sealing membrane extending around the opening. The tissue-interfacing attachment may comprise a cosmetics applicator. The cosmetics applicator may comprise a nail polish applicator, a mascara applicator, an eyebrow applicator, a lipstick applicator, or a powder applicator. The cosmetics applicator may comprise a lumen configured to be in fluid communication with the housing.

In some examples, a method for increasing surface energy of a portion of a body comprises generating a surface energy enhancing fluid using a gas generator; delivering the surface energy enhancing fluid to a surface of the portion of the body placed in a substantially sealed chamber, wherein the surface energy enhancing fluid may be configured to increase the surface energy of the portion of the body by 10 mN/m to 20 mN/m; and terminating the generating and/or delivering of the surface energy enhancing fluid after a duration.

The duration may be between 15 seconds and 35 seconds. The method may comprise recirculating remaining surface energy enhancing fluid within the substantially sealed system after the duration. The method may comprise absorbing remaining surface enhancing fluid using a filter. The filter may comprise activated charcoal. The surface energy enhancing fluid may comprise ozone. The generating the surface energy enhancing fluid may comprise applying an electrical current to oxygen in atmospheric air.

In some examples, a method of improving cosmetics application comprises placing a body part of a user in a substantially sealed chamber, wherein a surface energy enhancing fluid may be delivered to a surface of the body part and may increase surface energy of the surface of the body part by 10 mN/m to 20 mN/m for a duration; removing the body part from the chamber; and applying cosmetics to the surface of the body part within 3 minutes of removing the body part from the chamber.

The body part may comprise at least a portion of a face of the user. The body part may comprise a nail of the user. The body part may comprise a hair of the user. The surface energy enhancing fluid may comprise ozone.

In some examples, a method of increasing surface energy of a portion of a body comprises delivering a surface energy enhancing fluid to the portion of the body, wherein the surface energy enhancing fluid may be configured to increase the surface energy of the portion of the body; and stopping delivering the surface energy enhancing fluid after a duration.

The duration may be between 15 seconds and 35 seconds. The duration may be 30 seconds. Stopping delivering the surface energy enhancing fluid may comprise stopping generating the surface energy enhancing fluid. Stopping delivering the surface energy enhancing fluid may comprise redirecting a flow of the surface energy enhancing fluid. The method may further comprise circulating the surface energy enhancing fluid within a substantially sealed compartment. The substantially sealed compartment may comprise a sealing membrane configured to substantially seal the compartment upon applying the portion of the body to the compartment. The sealing membrane may be configured to inhibit the surface energy enhancing fluid from exiting the compartment. A concentration of the surface energy enhancing fluid may increase after every circulation of the surface energy enhancing fluid. The method may further comprise absorbing the surface energy enhancing fluid using a filter after the duration. The filter may comprise activated charcoal. The method may comprise increasing the surface energy of the portion of the body by 10 mN/m to 20 mN/m. The method may comprise outputting an alarm a second duration after the duration. The second duration may be between 1 min and 4 min. The second duration may be 3 min. The alarm may comprise an audible and/or visual alarm. The surface energy enhancing fluid may comprise ozone.

In some examples, a method of increasing surface energy of a portion of a body comprises delivering a surface energy enhancing fluid to the portion of the body from a delivery device for a duration and increasing the surface energy of the portion of the body by 10 mN/m to 20 mN/m.

The method may further comprise circulating the surface energy enhancing fluid within a substantially sealed compartment. The substantially sealed compartment may comprise a sealing membrane configured to substantially seal the compartment during delivering the surface energy enhancing fluid to the portion of the body. The sealing membrane may be configured to inhibit the surface energy enhancing fluid from exiting the compartment. A concentration of the surface energy enhancing fluid may increase after every circulation of the surface energy enhancing fluid. The method may further comprise absorbing the surface energy enhancing fluid using a filter after terminating the generating of the surface energy enhancing fluid. The filter may comprise activated charcoal. The method may further comprise generating the surface energy enhancing fluid. Generating the surface energy enhancing fluid may comprise generating the ozone by applying an electrical current to oxygen. The oxygen may be from air. The method may further comprise terminating the generating of the surface energy enhancing fluid after a duration, wherein the generated surface energy enhancing fluid may be eliminated within 1 minute to 5 minutes. The surface energy enhancing fluid may be eliminated within 3 minutes. Terminating the generating of the surface energy enhancing fluid may comprise deactivating a gas generator. The duration may be between 15 seconds and 35 seconds. The duration may be 30 seconds. The method may comprise outputting an alarm a second duration after the duration. The second duration may be between 1 min and 4 min. The second duration may be 3 min. The alarm may comprise an audible and/or visual alarm. The surface energy enhancing fluid may comprise ozone. The method may comprise pressurizing the surface energy enhancing fluid using a blower. The portion of the body may comprise at least a portion of a face. The portion of the body may comprise a nail. The portion of the body may comprise hair.

In some examples, a method of improving cosmetics application comprises increasing a surface energy of a body part by a predetermined amount and applying cosmetics to a surface of the body part within 3 minutes after increasing a surface energy of a body part.

The cosmetics may comprise nail polish. The cosmetics may comprise a powder. The cosmetics may comprise a lotion or cream. The cosmetics may comprise mascara. The cosmetics may comprise a foundation or primer. The cosmetics may comprise eye shadow. The cosmetics may comprise blusher. The cosmetics may comprise a concealer. The cosmetics may comprise a highlighter or luminizer. The cosmetics may comprise an eyebrow pencil, powder, or liquid. The cosmetics may comprise an eyeliner pencil, powder, or liquid. The cosmetics may comprise a lipstick or lip liner. The method may further comprise placing the surface of the body part in a substantially sealed chamber. The predetermined amount may comprise 10 mN/m to 20 mN/m. The method may further comprise generating a surface energy enhancing fluid using a gas generator. The method may further comprise deactivating the gas generator. The gas generator may be configured to generate the surface energy enhancing fluid between 0.2 g/hour and 0.8 g/hour. The gas generator may be configured to generate the surface energy enhancing fluid between 0.6 g/hour. The gas generator may be configured to generate the surface energy enhancing fluid by applying an electrical current to oxygen. The oxygen may be from air. The deactivating may be performed a duration after the increasing. The duration may be between 15 seconds and 35 seconds. The duration may be 30 seconds. The method may comprise outputting an alarm a second duration after the duration. The second duration may be between 1 min and 4 min. The second duration may be 3 min. The alarm may comprise an audible and/or visual alarm. The method may further comprise circulating the surface energy enhancing fluid within a substantially sealed compartment. The substantially sealed compartment may comprise a sealing membrane configured to substantially seal the compartment upon applying the surface of the body portion to the compartment. The sealing membrane may be configured to inhibit the surface energy enhancing fluid from exiting the compartment. The surface energy enhancing fluid in the compartment may be eliminated within 3 minutes of the increasing of the surface energy. A concentration of the surface energy enhancing fluid may increase after every circulation of the surface energy enhancing fluid. The method may further comprise absorbing the surface energy enhancing fluid using a filter after the increasing of the surface energy. The filter may comprise activated charcoal. The surface energy enhancing fluid may comprise ozone. The method may comprise pressurizing the surface energy enhancing fluid using a blower.

In some examples, a modular cosmetics applicator for increasing surface energy of body tissue comprises an inlet configured to be in fluid communication with a gas generator configured to generate a surface energy enhancing fluid so as to receive the surface energy enhancing fluid; a tissue interface portion configured to interface with the body tissue; and a lumen in fluid communication with the inlet and the tissue interface portion. The surface energy enhancing fluid received at the inlet may be configured to flow through the lumen to the tissue interface portion interfaces increase the surface energy of the body tissue.

The tissue interface portion may comprise a sealing membrane configured to substantially seal the applicator. The sealing membrane may be configured to inhibit the surface energy enhancing fluid from exiting the applicator. The applicator may comprise a brush. The applicator may comprise a powder or cream applicator. The applicator may comprise a lipstick applicator. The applicator may comprise a face mask. The tissue interface portion may comprise a perimeter of the face mask. A sealing membrane may extend around the perimeter of the face mask. The face mask may comprise a respiration opening. The surface energy enhancing fluid may comprise ozone. The inlet may be removably connectable to a gas generator.

In some examples, a cosmetic appliance configured to increase surface energy of body tissue before application of cosmetics to the body tissue comprises a gas generator configured to generate a surface energy enhancing fluid; an outlet configured to be coupled to a cosmetics applicator; and a lumen in fluid communication with the gas generator and the outlet. The surface energy enhancing fluid generated by the gas generator may be configured to flow through the lumen to the outlet.

The appliance may further comprise a recirculation path to recirculate the surface energy enhancing fluid. A concentration of the surface energy enhancing fluid may increase after recirculation of the surface energy enhancing fluid. The gas generator may be configured to generate the surface energy enhancing fluid between 0.2 g/hour and 0.8 g/hour. The gas generator may be configured to generate the surface energy enhancing fluid between 0.6 g/hour. The surface energy enhancing fluid may comprise ozone. The gas generator may be configured to generate the ozone by applying an electrical current to oxygen. The oxygen may be from air.

In some examples, a device configured to temporarily increase surface energy of body tissues may comprise a gas generator configured to generate a surface energy enhancing fluid configured to increase the surface energy of the body tissues, a gas chamber configured to provide exposure of the body tissues to the generated surface energy enhancing fluid, a first conduit coupled to the gas generator and the gas chamber, the first conduit configured to deliver the generated surface energy enhancing fluid from the gas generator to the gas chamber; and a second conduit coupled to the gas generator and the gas chamber, the second conduit configured to recirculate remaining surface energy enhancing fluid from the gas chamber to the gas generator. The gas generator, the gas chamber, the first conduit, and the second conduit may form a substantially sealed pathway for the surface energy enhancing fluid during use of the device on the body tissues sealed to inhibit the surface energy enhancing fluid from exiting the device.

The device may further comprise a blower configured to pressurize the generated surface energy enhancing fluid. A concentration of the surface energy enhancing fluid may be configured to increase after every circulation of the surface energy enhancing fluid through the second conduit. The device may further comprise a filter configured to absorb the surface energy enhancing fluid when the device is not in use. The filter may comprise activated charcoal. The device may further comprise a controller configured to deactivate the gas generator after a duration. The duration may be 10 seconds to 35 seconds. The gas generator may be configured to generate the surface energy enhancing fluid at 0.6 g/hour. The surface energy enhancing fluid may comprise ozone. Ozone may be generated by applying an electrical current to oxygen in the atmospheric air. The gas chamber may comprise a face mask. The face mask may comprise a sealing membrane extending around a perimeter of the face mask. The face mask may comprise an opening configured to let a mouth of a user breathe through the opening when the device is in use. The first conduit and the second conduit may be coupled to the face mask adjacent to each other. The first conduit and the second conduit may be coupled to the face mask at substantially opposing ends of the face mask. The gas chamber may comprise a plurality of openings configured to receive a plurality of digits. Each of the plurality of openings may be covered by a sealing membrane. The sealing member may comprise a slit to receive a digit. The gas chamber may be located in a cosmetics applicator. The cosmetics applicator may comprise a nail polish applicator, a mascara applicator, an eyebrow applicator, a lipstick applicator, or a powder applicator. The gas chamber may comprise a lumen located in the cosmetics applicator.

### BRIEF DESCRIPTION OF THE DRAWINGS

FIGS. 1A-1C illustrate schematically different states of wedding when a drop of liquid contacts a solid substrate.

FIGS. 2A-2C illustrate schematically different failure modes when a coating is applied to a substrate.

FIG. 2D illustrates an example chemical reaction catalyzed by ozone in which a carbon double bond is broken.

FIG. 3 illustrates schematically an ozone generator for generating ozone from air.

FIGS. 4A and 4B illustrate schematically a nail priming device using ozone as an adhesion promoter.

FIG. 5A illustrates a front view of an example nail priming device using ozone as an adhesion promoter.

FIG. 5B illustrates a top view of the nail priming device of FIG. 5A.

FIG. 5C illustrates a bottom view of the nail priming device of FIG. 5A.

FIG. 5D illustrates a right view of the nail priming device of FIG. 5A.

FIG. 5E illustrates a back view of the nail priming device of FIG. 5A.

FIG. 5F illustrates a left view of the nail priming device of FIG. 5A.

FIG. 5G illustrates a perspective view of the nail priming device of FIG. 5A.

FIG. 5H illustrates an internal view of the nail priming device of FIG. 5A.

FIG. 5I illustrates a perspective view of a housing of the nail priming device of FIG. 5A.

FIG. 5J illustrates a partially exploded view of the nail priming device of FIG. 5A.

FIG. 5K illustrates a bottom perspective view of an ozone flow path of the nail priming device of FIG. 5A.

FIG. 5L illustrates a top perspective view of the ozone flow path of the nail priming device of FIG. 5A.

FIG. 6 illustrates a scanning electron microscopic view of an example fingernail before being treated by ozone.

FIG. 7 illustrates schematically a face priming device using ozone as an adhesion promoter.

FIG. 8A illustrates a right view of an example generator chamber of a face priming device using ozone as an adhesion promoter.

FIG. 8B illustrates a top view of the generator chamber of FIG. 8A.

FIG. 8C illustrates a bottom view of the generator chamber of FIG. 8A.

FIG. 8D illustrates a front view of the generator chamber of FIG. 8A.

FIG. 8E illustrates a back view of the generator chamber of FIG. 8A.

FIG. 8F illustrates a left view of the generator chamber of FIG. 8A.

FIG. 9A illustrates a front view of an example face mask of a face priming device using ozone as an adhesion promoter.

FIG. 9B illustrates a back view of the face mask of FIG. 9A.

FIG. 9C illustrates a bottom view of the face mask of FIG. 9A.

FIG. 9D illustrates a top view of the face mask of FIG. 9A.

FIG. 9E illustrates a right view of the face mask of FIG. 9A.

FIG. 9F illustrates a left view of the face mask of FIG. 9A.

FIG. 10 illustrates a magnified view of an example skin sample.

FIG. 11 illustrates a microscopic view of an example skin sample.

FIGS. 12A and 12B illustrate perspective views of an example face priming device using ozone as an adhesion promoter including a mask and a generator chamber.

FIGS. 12C and 12D illustrate perspective views of the generator chamber of FIGS. 12A and 12B.

FIGS. 12E and 12F illustrate perspective views of the mask of FIGS. 12A and 12B including an adapter.

FIGS. 12G and 12H illustrate perspective views of the adaptor of FIGS. 12E and 12F.

FIG. 13A illustrates an example face priming device using ozone as an adhesion promoter including a mask and a generator chamber.

FIGS. 13B and 13C illustrate perspective views of the generator chamber of FIG. 13A.

FIG. 14 illustrates a perspective view of an example face priming device using ozone as an adhesion promoter including a mask and a generator chamber with tubing connecting the face mask and the generator chamber being omitted for clarity.

FIG. 15A illustrates a perspective view of an example face priming device using ozone as an adhesion promoter including a mask and a generator chamber with tubing connecting the face mask and the generator chamber being omitted for clarity.

FIG. 15B illustrates a front view of a face mask of the face priming device of FIG. 15A applied to a user's face.

### DETAILED DESCRIPTION

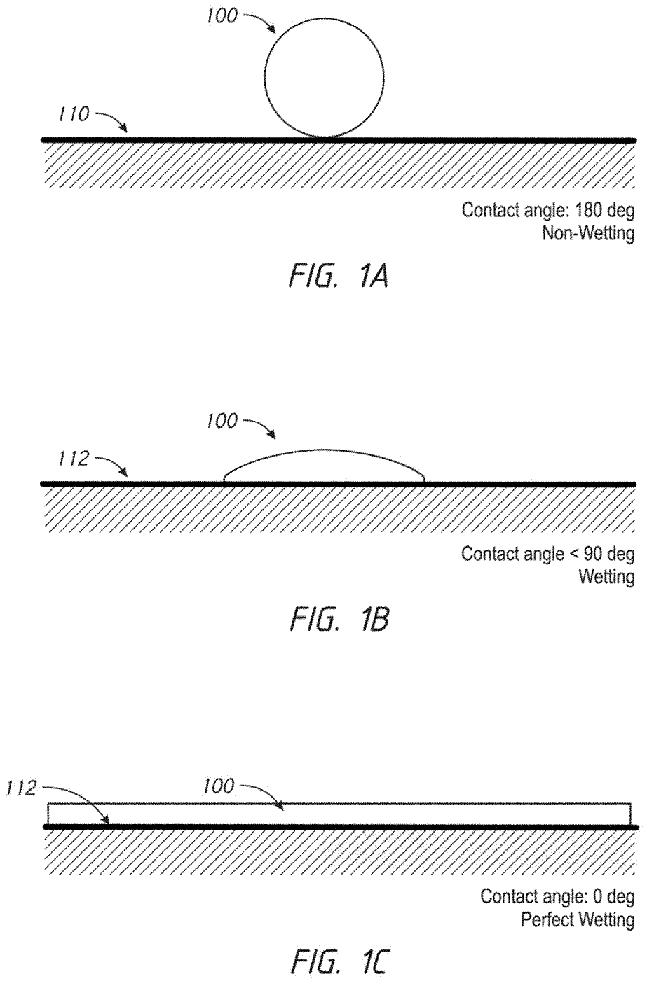

Cosmetics are normally applied to biological tissues on the outside of the body. These tissues, like all materials, have a property that is used to define how well liquids or liquid-like coatings or oils sticks or paints to it, called the surface energy. FIGS. 1A-1C illustrate various states of wetting when a drop of liquid 100 contacts a solid substrate 110, 112. If the surface energy of the substrate 110 does not change upon the addition of the drop 100, that is, the contact angle is 180 degree, the substrate 110 is said to be non-wetting, such as shown in FIGS. 1A. If the surface energy of the substrate 112 changes upon the addition of the drop 100, the substrate 112 is said to be wetting, such as shown in FIGS. 1B and 1C. The higher the surface energy, the more evenly and smoothly the applied coating adheres, such as the state of perfect wetting shown in FIG. 1C, in which the contact angel is 0 degree or substantially 0 degree, compared to the state of wetting shown in FIG. 1B, in which the contact angle is greater than 0 degree but less than 90 degree.

An example surface 110 in FIG. 1A can be a low energy surface like Teflon. An example surface 112 in FIG. 1B can be a paintable plastic or a wood. An example surface 112 in FIG. 1C can be a high surface energy material like metal or glass.

The surface energy of a material is an inherent property of that material and does not tend to change without treatments on the surface of the material. Some typical values for surface energy (from lower to higher) can include: (mN/m)

| | Surface Energy |
|---|---|
| Material | (mN/m) |
| Teflon | 20 |
| Wax | 26 |
| Skin/Hair/Nails | 27 to 30 |
| Rubber | 34 |
| Non-Paintable Plastics | 25 to 36 |
| Paintable Plastics | 37 to 50 |
| Wood | 200 |
| Glass | 290 |
| Aluminum | 500 |
| Copper | 1,000 |

Some example surface energy levels for proper bonding can include:

| | Surface Energy |
|---|---|
| Material | (mN/m) |
| Water-Based Inks | 48 to 56 |
| Paints | 36 to 52 |
| Water-Based Glue | 48 to 56 |

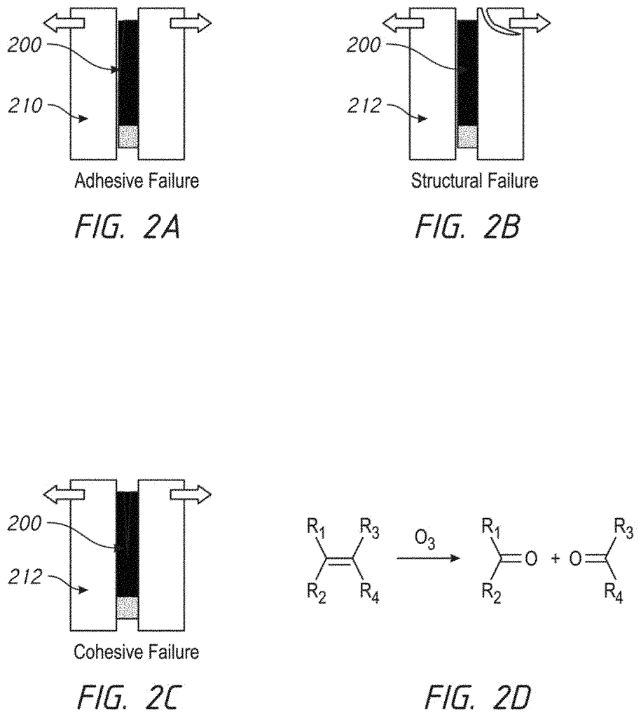

As shown in FIG. 2A, when a lower surface energy substrate 210 is painted, it can be subject to adhesive failure. The bond between the substrate 210 and the coating 200 fails at the boundary between the coating 200 and one of the substrates 210. In contrast, bonding of higher surface energy materials 212 can fail at the substrate material 212 (FIG. 2B) and/or in the coating 200 (FIG. 2C). In FIGS. 2B and 2C, the bond between the coating 200 and the substrates 212 is stronger than the materials in question, that is, the substrates 212 and the coating 200. Adhesive and cohesive failures are listed in ASTM B571 as an example.

Due to the lower surface energy of the biological body tissue, including but not limited to skin, hair, and fingernails, these biological body tissues are more difficult for cosmetics to stick to than a material of a higher surface energy, at least for a substantial duration such as throughout a day. Cosmetics tend to form adhesive bonds with the skin, hair, fingernails, and the like. As a result, the nail polish can flake off any time after the nail polish is applied, the dye can wash out over time, and the foundation can clump and/or flake off during the course of a day.

Treatments to increase the surface energy of a material at the surface of a material are available. However, most of the currently available treatments are too harsh for use on living tissue. These currently available treatment methods include, for example, flame treating, corona or electrical shock treating, and liquid chemical treating.

The present disclosure provide a safer and/or more effective way to increase the surface energy of a living tissue that can include using a gas or aerosol. A device can use gas or aerosol methods (both gas and aerosol may be referred to as gas going forward) to improve the surface energy of skin, hair (including facial and/or body hair), and nails (fingernails and toenails) safely and effectively.

The gas that can improve the surface energy of a solid substrate is known as an adhesion promoter or surface energy enhancing gas. One example of an adhesion promoter gas is ozone. Ozone can act as a catalyst to break olefinic or double bonds in carbon based compounds. A chemical reaction catalyzed by ozone is shown in an equation in FIG. 2D. Additional examples of an adhesion promoter or compounds that can enhance surface energy can include, for example, Vinyl Amine/Vinyl Alcohol Copolymer, 4-(3-Aminopropyl) morpholine, 4-(γ-Aminopropyl) morpholine, N-Aminopropyl-morpholine, N-(3-Aminopropyl) morpholine, Aminopropylsilanol, Aminopropylsiloxanes, N-3-Aminopropyl-N-tallow alkyl trimethylene diamines, Ammonium lauryl sulfate, Cocamidopropyl hydroxysultaine, Cocamidopropyl PG-dimonium chloride phosphate, C10-12 pareth-6, C10-12 pareth-7, C10-12 pareth-8, C12-14 pareth-3, C13-15 pareth-5, C13-15 pareth-9, C14-15 pareth-3, Disodium cocoamphodiacetate, Hydroxylated lecithin, Isodeceth-5, Lauryl glucoside, Oleth-10, Palmitamine oxide, PEG-8 dilaurate, PPG-10 cetyl ether, Sodium C12-15 pareth-7 sulfonate, Sodium trideceth sulfate, Sucrose distearate, Sucrose stearate, and liquid adhesive (such as the Mastisol® Medical Liquid Adhesive from Eloquest Healthcare, Inc.). Other suitable adhesion promoter fluid can also be incorporated into any of the example apparatuses and methods for increasing the surface energy of a solid substrate.

At appropriate concentrations, ozone or another adhesion promoter can quickly break the bonds of skin, hair, and nails, which contain amino acids with double bonds, and increase the surface energy of the skin, hair, and nails, for example, by between 10 mN/M and 20 mN/M in the case of ozone. Ozone or another adhesion promoter produced by such devices does not permeate the skin and is not intended to be in sufficient concentration to act as a disinfectant.

The treatment of skin, hair, and nails by using priming devices to apply ozone or other adhesion promoter in proper concentration and/or for a sufficient duration can raise the surface energy of the skin, hair, nails, and the like, by about 10 to about 20 mN/m, for example, from between about 27 mN/m and about 30 mN/m to between about 47 mN/m and about 50 mN/m, by creating open charged chemical bonds. The open charged chemical bonds last temporarily until oxygen is attracted to the bonds and the surface energy returns to its original value. A user can apply cosmetics to the skin, hair, and/or nails before oxygen is attracted to the bonds. Accordingly, when cosmetics are applied immediately after treatment, the priming devices can increase the effectiveness of cosmetics, making paints, dyes and/or other cosmetic binders form cohesive bonding rather than adhesive bonding with the skin, hair, nails, and the like. This change of bonding type can improve the performance of all cosmetics. For example, the nail polish may chip less, the hair dye and skin tanning coatings may last longer, and the foundation, lipstick, and/or mascara may last longer and may be more comfortable to wear with less clumping and flaking.

### Ozone Generation Destruction

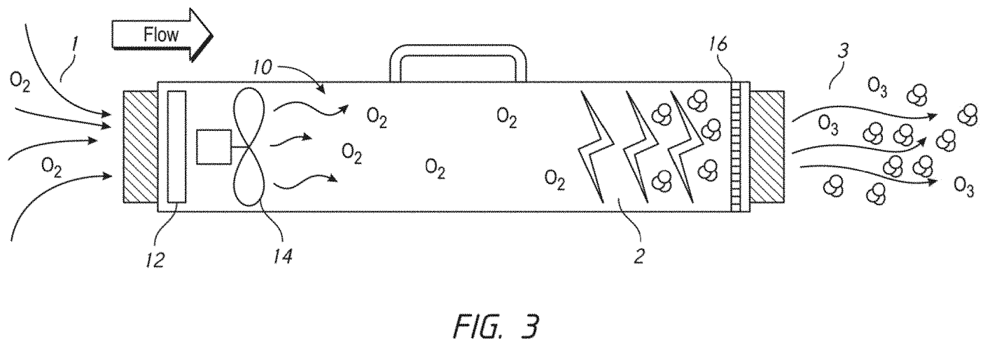

Generation of ozone will be described below with reference to FIG. 3. The example priming devices disclosed herein can incorporate any ozone generator (such as standard ozone generators) or any electric system (such as static electric systems), in which ozone functions as the medium.

At step 1, ambient air, which include oxygen molecules, can be pulled into an enclosed chamber 10 (also known as a corona cell) of the ozone generator at one end of the chamber 10. A filter 12 can prevent dust and/or insects from entering or logging in the chamber 10. A fan 14 can accelerate air through the ozone generator to another end of the chamber 10. At step 2, ozone can be generated from high voltage electrical sparks generated via a high voltage corona mechanism within the chamber 10, which is a controlled environment. Typical generation amounts can range from about 0.05 g/hour to over about 10 g/hour for commercial applications (such as in hot tubs and air cleaners). Industrial units can generate nearly unlimited amounts of ozone based on use in water treatment and other high volume applications. As the use of ozone increases, the requirement of power supply and the weight of the generator increase. A protective stainless steel grill 16 can be place near the other end of the chamber 10 to ensure that a user does not touch the chamber while the ozone generator is in operation. At step 3, the generated ozone can exit the other end of the chamber 10. The ozone generation process can take less than one second.

Although zone is not harmful to the human, exposure to ozone may cause discomfort, with certain individuals being more sensitive to ozone than others. The discomfort can include a tickling sensation in the back of the throat or any epithelial lining, the unpleasant odor of the ozone, temporary irritation to the eyes, and/or otherwise. Ozone may also cause degradation of plastic and/or rubber components upon exposure. Ozone can also additionally contribute to environmental pollution, such as by contributing to more smog formation. As a result, the devices incorporating an ozone generator may also include mechanisms to contain the ozone and effectively and quickly destroy any remaining ozone after use. Accordingly, the devices disclosed herein can include sealing members to reduce and/or prevent leakage of ozone to the environment, recirculation pathways (for example, with one-way valves or otherwise) for ozone to reduce the amount of ozone needed for priming a surface on a user, and/or appropriate filter for absorbing the ozone after use.

### Examples of a Nail Priming Device

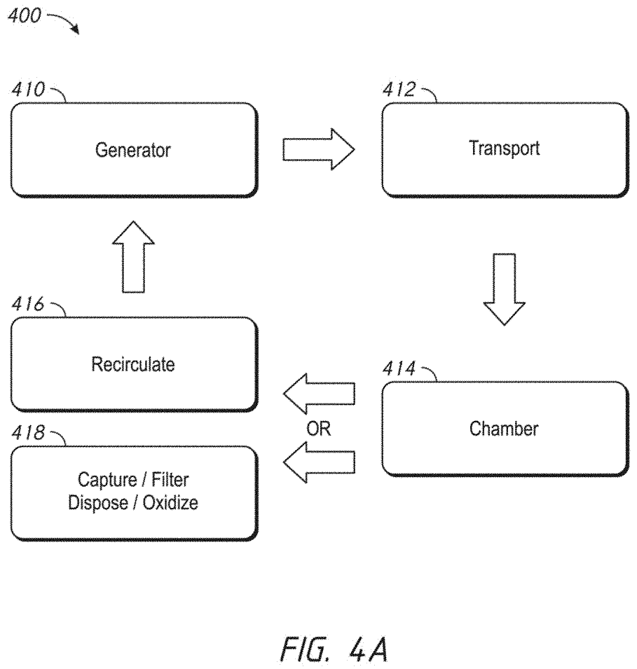

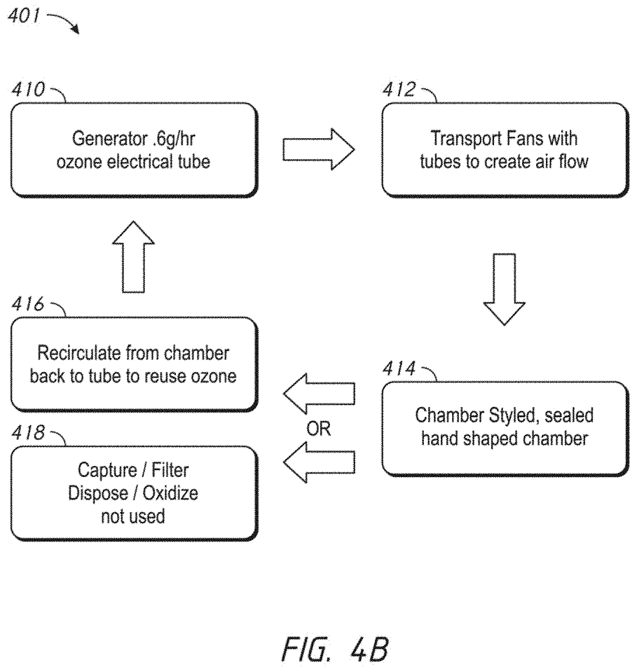

A nail priming device can apply an adhesion promoter to the finger and/or toe nails prior to application of a nail polish. As shown in FIGS. 4A and 4B, a nail priming device 400, 401 can include a module for creating a surface-energy enhancing gas or aerosol (gas generator 410), ways of transporting 412 the gas or aerosol (such as using a fan, a blower, or other pressure based transport method), a sealed or semi-sealed chamber 414 in which the tissue is exposed to the gas or aerosol for a limited amount of time (such as a chamber having a hand shape as shown in FIG. 4B), a recirculation system 416, and/or a filter system 418.

The device 400, 401 can be powered by being plugged into a socket, or optionally include a battery. The battery can have a power of about 60 W, or about 75 W, or about 90 W, or about 105 W, or about 120 W, or any range between such values. As shown in FIG. 4B, the generator 412 of the device 401 can produce ozone at a rate of about 0.6 g/hour. The generator 412 can optionally include an electrical tube (such as one similar as shown in FIG. 3) for producing ozone from ambient air.

The flow of ozone can move from the chamber 414 to either a recirculation system 416 or a filter system 418 or both. The recirculation system 416 can return the gas or aerosol to the chamber 412, or to the electrical tube in FIG. 4B for reuse. The filter or other similar mechanism 418 can clean and/or store the remaining gas or aerosol. In some examples, the flow can be directed to the recirculation system 416 for a predetermined number of time, and/or for a predetermination duration, before being directed to the filter 418. In some examples, such as in the device 401 of FIG. 4B, the filter can oxidize, dispose, and/or otherwise destroy the unused ozone.

In some examples, a nail priming device can include a housing, an ozone generator, a finger chamber, hoses for the gas to circulate, a high voltage transformer, a power button, and a timer circuit board. In some examples, the ozone generator can generate ozone at a rate of about 0.1 g/hour to about 2.0 g/hour (e.g., about 0.1 g/hour, about 0.2 g/hour, about 0.4 g/hour, about 0.6 g/hour, about 0.8 g/hour, about 1.0 g/hour, about 1.5 g/hour, about 2.0 g/hour, and ranges between such values). The user can place the fingers into the finger chamber of the nail priming device through holes or slits in a soft sheet of material and press the power button. The button can activate a timer device or a controller to activate the ozone generator and/or the circulation apparatus.

The fingers can be sealed by the soft membrane and the ozone can be generated at a desired or set concentration. In some examples, the soft membrane can include Neoprene, rubber (for example, ethylene propylene diene monomer (EPDM) rubber of Shore A durometer 40), or the like. In some examples, the soft membrane can have a thickness of about 0.5 mm, about 1 mm, about 2 mm, about 3 mm, and ranges between such values.

Fans in the circuit can pull the ozone from the generator and blow it through the finger chamber and over the nails. The device can include a closed system and ozone can be re-circulated back into the ozone chamber. The device can run a plurality of circulations of the ozone through the chamber, such as about 6 time, or about 9 times, or about 12 times, or about 15 times, or about 18 times, or any ranges between such values. In some examples, the ozone concentration can be increased after every revolution through the circuit. The closed system allows a smaller, cheaper, and lighter generation unit to create higher concentrations of ozone in a shorter period of time. Recirculation of ozone can optionally reduce the total amount of odor generated by the device.

The device can include a timer, such as a timer circuit board. After a sufficient period of time or duration, (for example, approximately 15 seconds, approximately 30 seconds, or approximately 45 seconds, or ranges between such values), the device can shut down automatically or the generation chamber can be deactivated. In some examples, the device can include a controller configured to deactivate the gas generator after the aforementioned duration. The ozone remains inside the chamber, where it oxidizes over time. This amount of oxidation time may be reduced for devices that see heavy use (such as commercial vs individual units), for example, by filtration using activated charcoal or carbon sheets or otherwise inside the machine. These sheets can be replaceable. In some examples, the ozone inside the chamber can be eliminated a second duration after the device stops generating ozone. The second duration can be about 1 minute, about 2 minutes, about 3 minutes, about 5 minutes, or ranges between such values. In some examples, the controller is configured to emit a signal after the second duration. The controller can be in communication with the timer circuit board. The signal can include an alarm (for example, an audible and/or visual alarm).

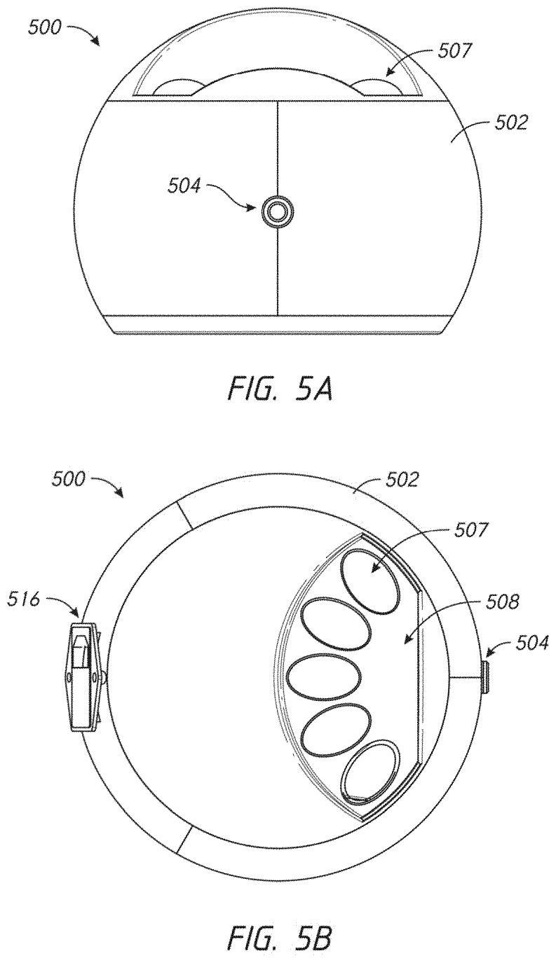

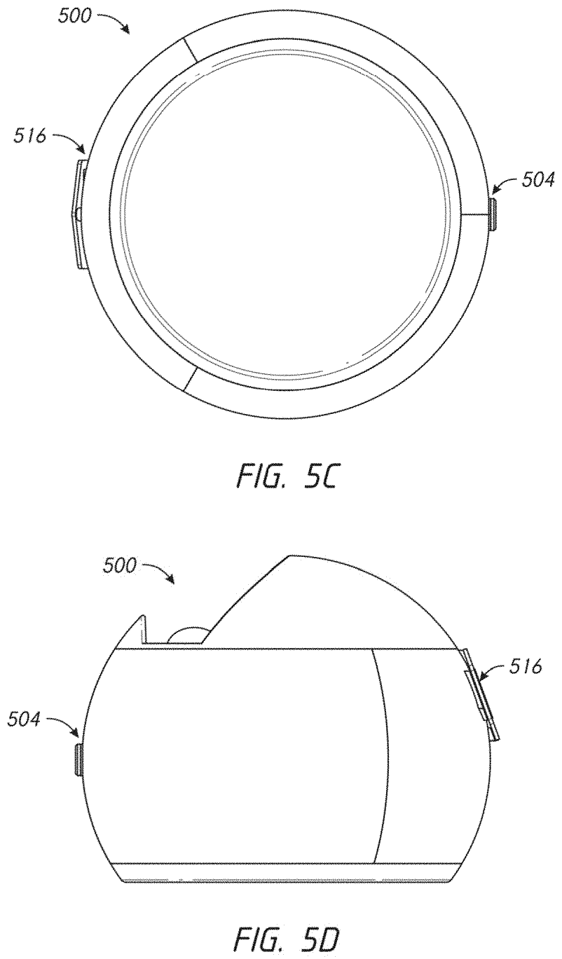

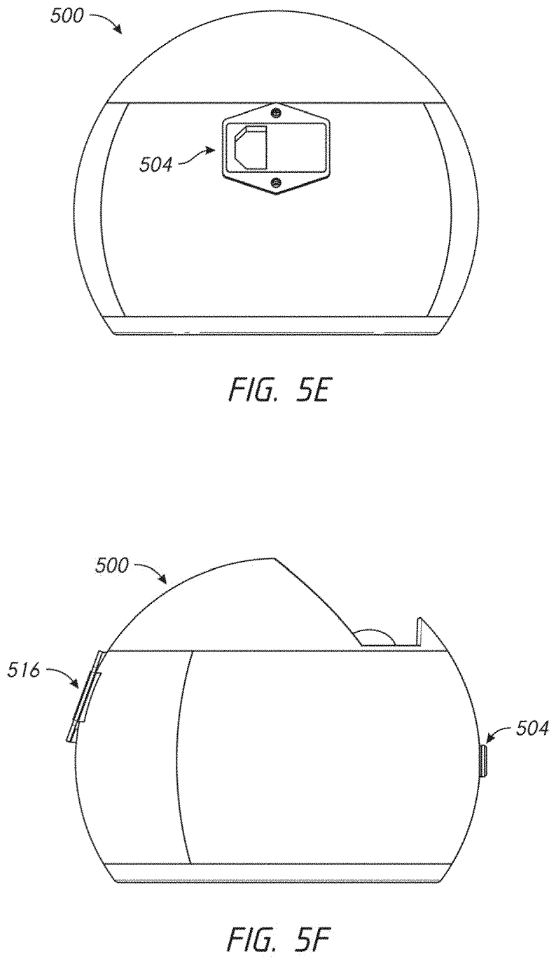

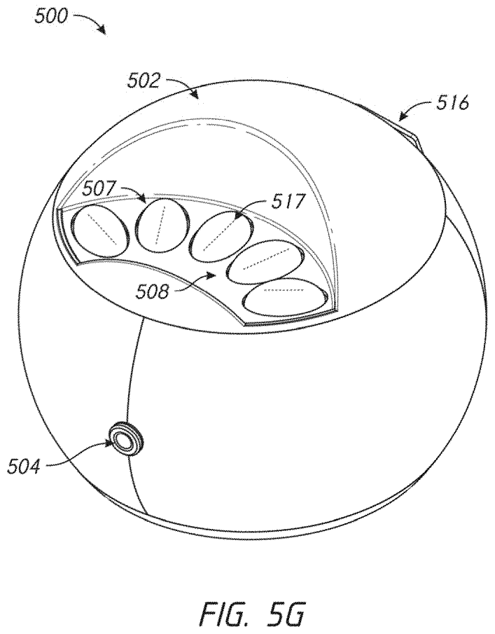

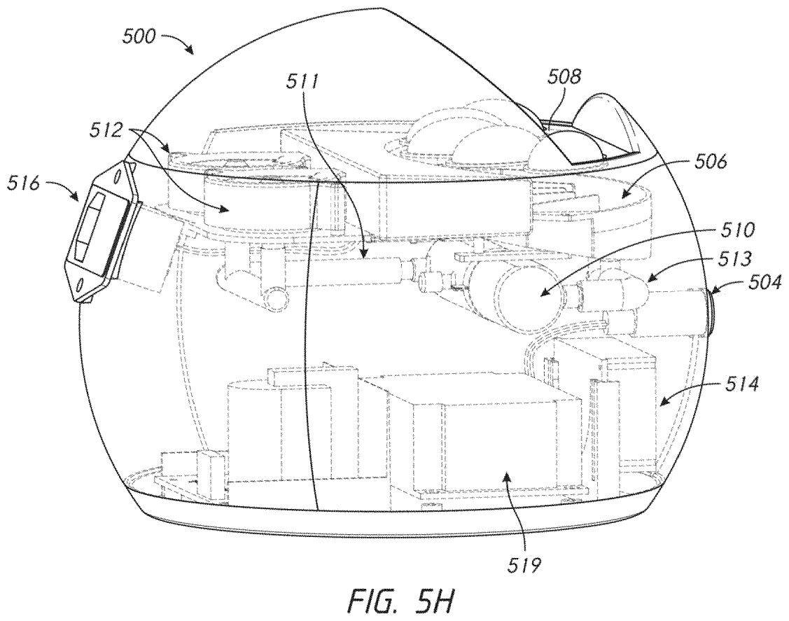

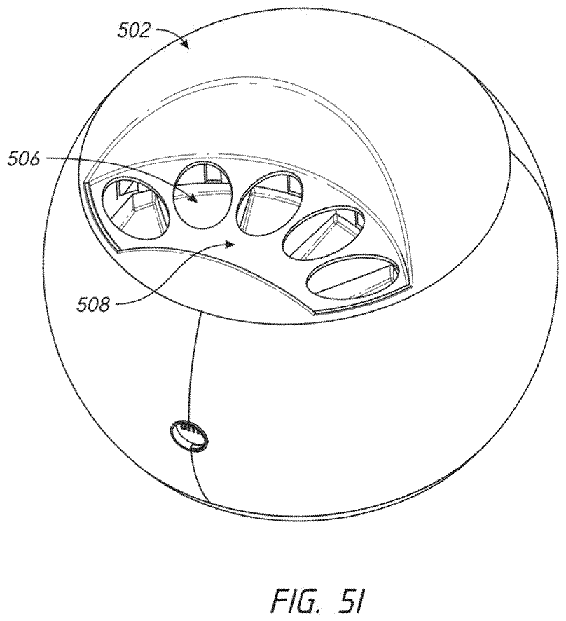

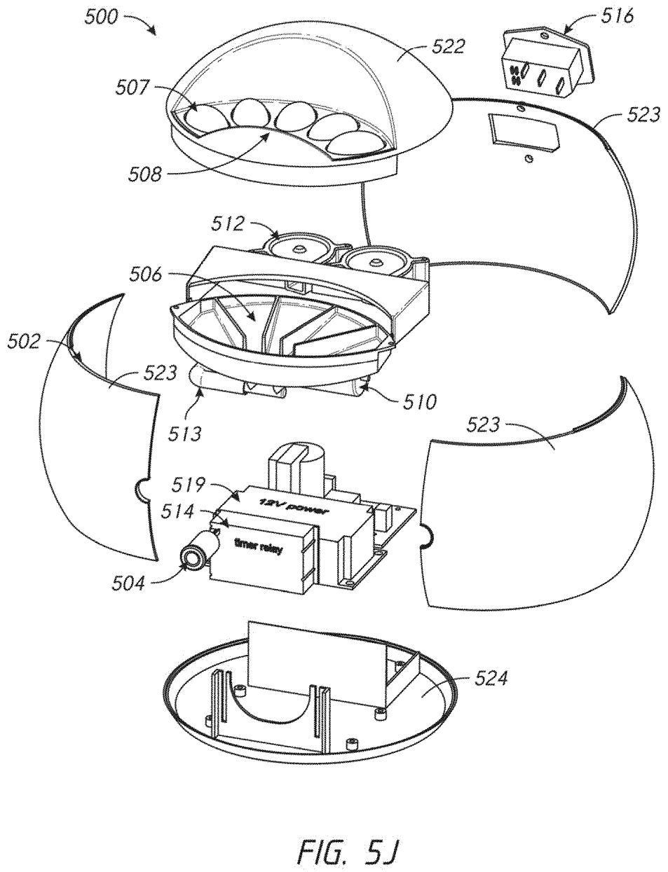

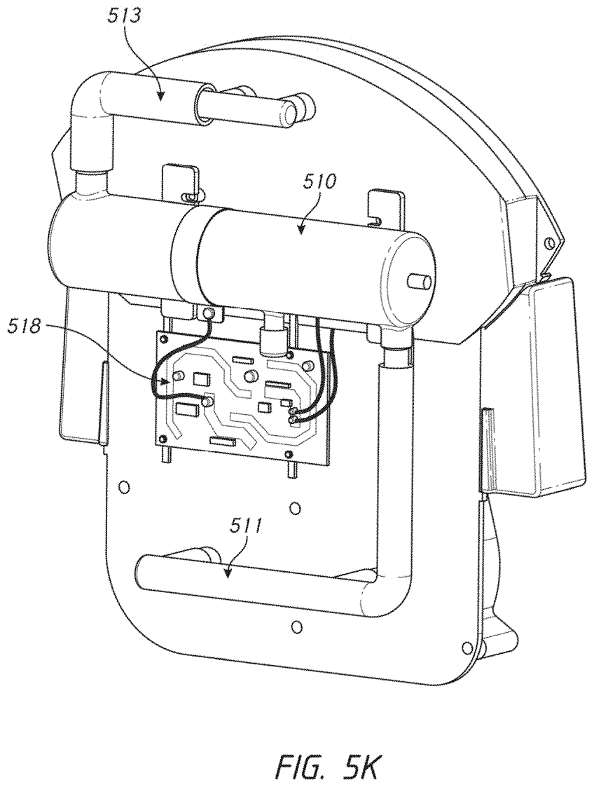

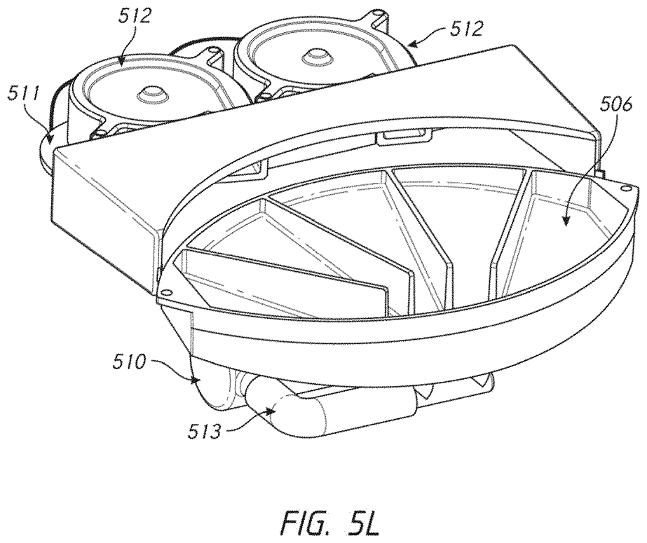

FIGS. 5A-5H and 5J illustrate various views of an example nail priming device 500. The device 500 can include any of the features of the device 400, 401 described above. Features of the device 500 can be incorporated into features of the device 400, 401 and features of the device 400, 401 can be incorporated into features of the device 500.

The nail priming device 500 can include a housing 502, such as shown in FIG. 5I. The housing 502 can have a generally circular outer shape, or any other shape. As shown in FIG. 5J, a plurality of side walls 523, a top wall 522, and a bottom wall 524 can be assembled together forming the housing 502. The top wall 522 can include the platform 508 and the openings into the finger chamber 506. The platform 508 can provide support for the user's hand when the user's fingers are inserted into the finger chamber 506 through the openings. Each of the openings can be covered by the soft and/or resilient sealing membranes 507. The membrane 507 can include a slit (shown by broken lines 517, which are omitted in FIGS. 5B, 5H, and 5J for clarity) for a finger to be inserted therethrough so that the finger chamber 506 is sealed or substantially sealed when the user's fingers are inserted into the openings. The finger chamber 506 can include individual compartments separated by partitions for each fingernail, such as shown in FIG. 5J, or include a chamber with a single compartment.

The housing 502 can have a size suitable to be portable and/or for being used on a tabletop. A power button 504 can be located on a front side of the device, as shown in FIG. 5A. The power button can also be located anywhere else on the device housing 502. The device 500 can include a power source or connection to a power source, for example, a power plug 516. The device 500 can also optionally include a battery power 519 such as shown in FIG. 5G. The battery can have a power of about 60 W, or about 75 W, or about 90 W, or about 105 W, or about 120 W, or any range between such values.

As shown in FIGS. 5H and 5J-5L, the housing 502 of the device 500 can enclose an ozone generator, such as in the form of an electrical tube 510. Fans or blowers 512 can pull the ozone generated from the tube 510 through the finger chamber 506 and over the nails inside the chamber 506. A timer circuit board 514 can also be enclosed by the housing 502. The device 500 can also include a controller 518 (see FIG. 5K) in communication with the time circuit board 514. The finger chamber 506 and the electrical tube 510 can be coupled with tubing 511, 513 for delivering ozone generated in the tube 510 to the chamber 506 (such as using the tubing 511) and for delivering the remaining ozone from the chamber 506 to the tube 510 (such as using the tubing 513) for recirculation. The location of the various components enclosed by the housing 502 can vary. The device 500 can also optionally include a generator of another adhesion promoter.

In the device 500, the membranes 507, the finger chamber 506, and the tubing 511, 513 can form a sealed pathway for the flow of ozone. The membrane 507 can prevent leakage of ozone from the gaps between the fingers or toes and the openings to the finger chamber 506. As described above, recirculating the remaining ozone can prevent ozone from leaking into the ambient air and/or reduce the total amount of ozone needed to reduce the surface energy of the nail surface. Optionally, the ozone concentration in the finger chamber can be increase after every circulation.

The controller 518, located on a printed circuit board as shown in FIG. 5L, can be in communication with the ozone generator 510 to activate and/or deactivate the ozone generator 510. In some examples, the ozone generator 510 is automatically switched off after a predetermined amount of time and/or after a predetermined number of re-circulations. The timer 514 that is in communication with the controller 518 can determine when to activate and/or deactivate the ozone generator 510.

When the user removes the hand from the nail priming device, the user may detect an ozone odor on the skin of the hand. While this smell is detectible, the polish can be applied. The user can have about 2 minutes, about 3 minutes, about 5 minutes, or ranges between such values, to paint the first coat of nail polish on the nails before the ozone is substantially oxidized by the air. This can be repeated for the other hand. The nail priming device can also be used to prime the toenails with ozone.

In testing, is has been shown by an ASTM B571 adhesion cross hatch pull-off testing that a basic nail polish that does not have an included chemical primer can change from adhesive failure without ozone treatment to cohesive failure with ozone treatment. Untreated test tape samples with nail polish applied showed delamination by flaking of the nail polish that remains on the test tape. Treated test tape samples showed no flaking. A laboratory bench test has been developed to simulate an impact to a painted nail designed to simulate hitting the painted nail against a hard surface, such as a chair or a set of keys. The testing showed that an untreated nail had approximately 50% more loss in the area of polish as compared to an ozone-treated nail for larger impacts. In addition, smaller chips that were observed on untreated nails were not detectible as chips on the treated nails. Rather, a marred surface may be observed on the nail polish without showing any portion of the substrate nail that has been ozone treated. For example, in certain samples, an impact of substantially the same force created an about 2-mm wide chip on an untreated nail, but an about 1 mm wide chip on an ozone treated nail.

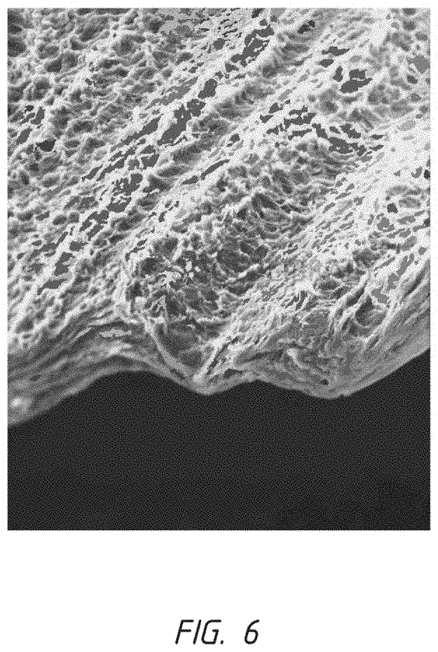

Visually, when comparing an ozone treated nail to an untreated nail, the glossiness of the ozone treated nail can be higher than the untreated nail. This can be attributed to the wetting phenomena. As can be seen in FIG. 6, an untreated nail surface is not smooth at the microscopic level. Untreated nails can leave gaps or pockets of air between the polish and the nail due to lack of wetting. When treated by ozone or another adhesion promoter, such as shown above in FIGS. 1B and/or 1C, the increased surface energy of the substrate nail can cause wetting to occur and pull the polish into the nail gaps so as to create a better surface finish and/or gloss level. The polish can also be more evenly distributed across the surface of the treated nail, with less variation in the cross sectional area of the polish. In effect, the nail polish can “shrink wrap” the ozone treated nail and result in a better buffed, shinier polished nail.

### Examples of a Face Device

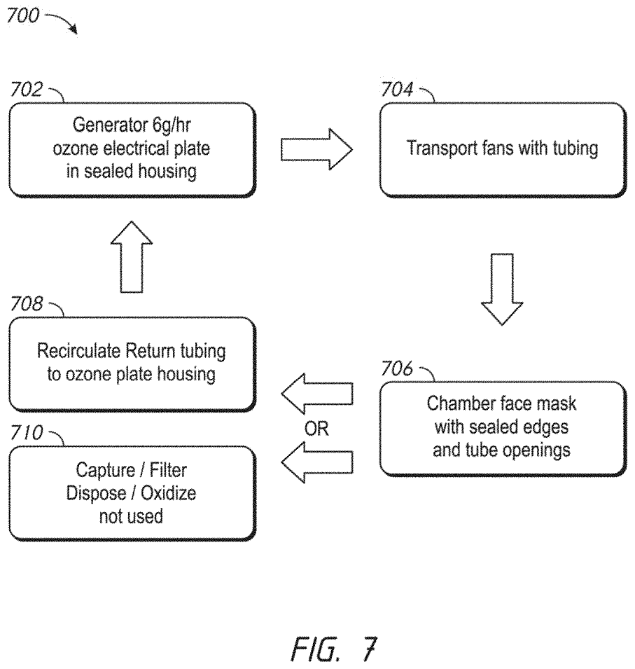

A user's face or any portion thereof can be primed with ozone or another adhesion promoter to increase the surface energy of the surface of the face or any portions thereof, thereby improving the bond between the makeup (or cosmetics) and the face or any portions thereof. FIG. 7 illustrates an example operation of a face priming device 700. The face priming device can use a module for creating a surface-energy enhancing or adhesion promoter (generator 702), ways of transporting the gas or aerosol using a fan or other pressure based transport method (blower and tubing 704), a sealed or semi-sealed mask in which the skin tissue is exposed to the gas or aerosol for a limited amount of time (mask 706), a recirculation system 708, and optionally a filter system 710. The recirculation system 706 can return the gas or aerosol to the mask. The optional filter system 708 can direct the flow to a filter or other mechanism that cleans, oxidizes, and/or stores the remaining gas or aerosol. The components in the face priming devices disclosed herein can have any of the features of the same or similar components in the nail priming device examples described above.

In some examples, a face priming device can include a housing, an ozone generator, a sealed face mask with hose interfaces, hoses or tubing for the gas to circulate, and a power switch. The fan device can also optionally include a high voltage transformer. The user can place the mask over the face and seal a soft membrane on the mask to the skin. The user can turn on the power switch for an appropriate amount of time. In some examples, the gas can be circulated for about 10 seconds, about 15 seconds, about 20 seconds, about 30 seconds, about 35 seconds, or ranges between such values. Additionally or optionally, the device can automatically turn off the power after the predetermined amount of time. In some examples, the ozone generator can generate ozone at a rate of about 0.1 g/hour, about 0.2 g/hour, about 0.4 g/hour, about 0.6 g/hour, about 0.8 g/hour, about 1.0 g/hour, about 1.5 g/hour, about 2.0 g/hour, and any ranges between such values.

Face skin can be sealed (for example, entirely sealed or at least substantially entirely sealed) inside the mask and sufficient clearance can be given between the face and the mask to allow appropriate circulation of the ozone gas. In some examples, the face skin can be sealed inside the mask such that leakage is below about 0.20 parts per million (PPM), about 0.15 PPM, about 0.10 PPM, about 0.09 PPM, about 0.08 PPM, about 0.05 PPM, about 0.01 PPM, and any ranges between such values. The ozone can be generated at an appropriate concentration. Fans or blowers in the ozone flow circuit can pull ozone from the generator chamber or housing and blow the ozone through the mask and over the face and all associated hair and lips. An opening in the mask can optionally be located at or near the mouth of the user for breathing during the ozone application. The ozone in the area between the face and mask can be in a closed or at least substantially closed system and the unused ozone can be re-circulated back into the ozone generator chamber. In some examples, the ozone concentration can be increased after every revolution through the circuit so as to reduce the total amount of ozone required for each use of the device. The closed system can allow a smaller, cheaper, and lighter generation unit to create higher concentrations of ozone in a shorter period of time. It can also reduce the odor generated by the device. When the application of ozone on the user's face is complete, the user can remove the mask. A small amount of the odor of the ozone may be released while the mask is being removed. However, most of the unused zone in the mask, chamber, and/or hoses while moving the mask (typically onto a flat tabletop surface) can be collected inside the chamber, where the ozone oxidizes over time. This amount of oxidation time may be further reduced for devices that see heavy use (such as commercial vs individual units), for example, by filtration using activated charcoal or carbon sheets or otherwise inside the chamber. These filters can be replaceable.

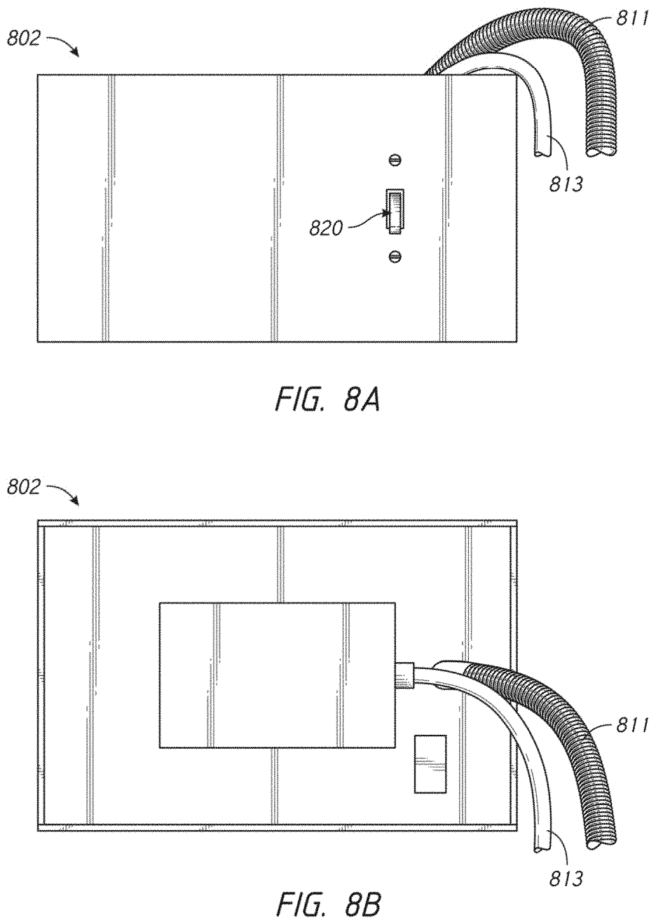

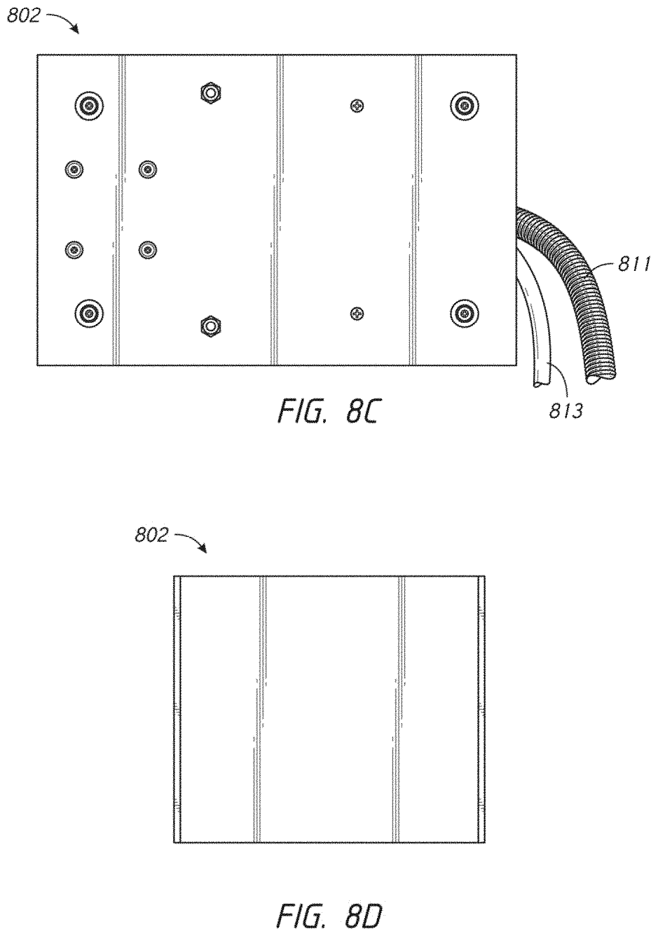

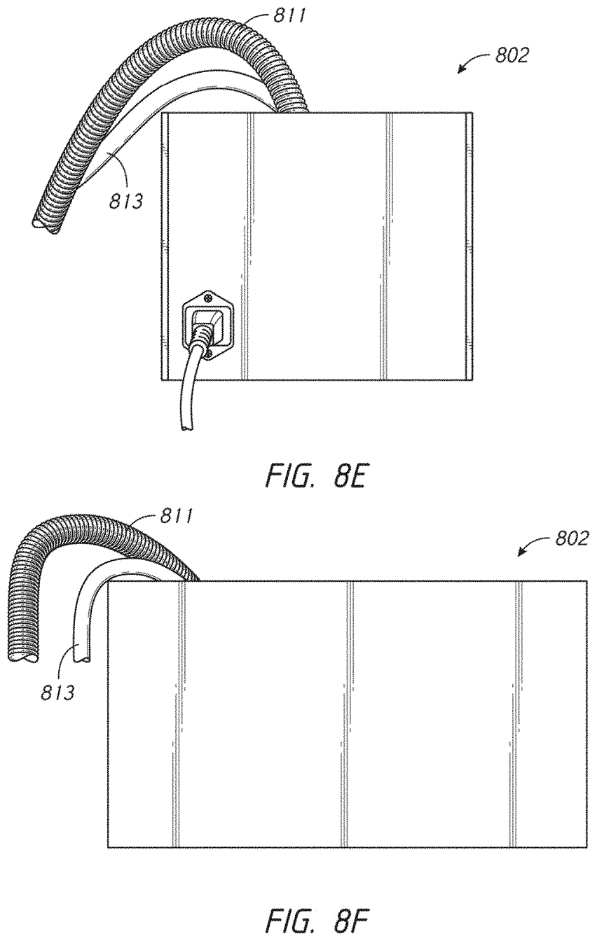

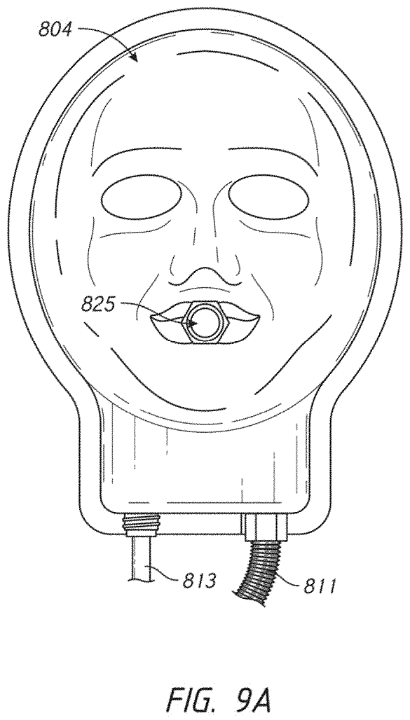

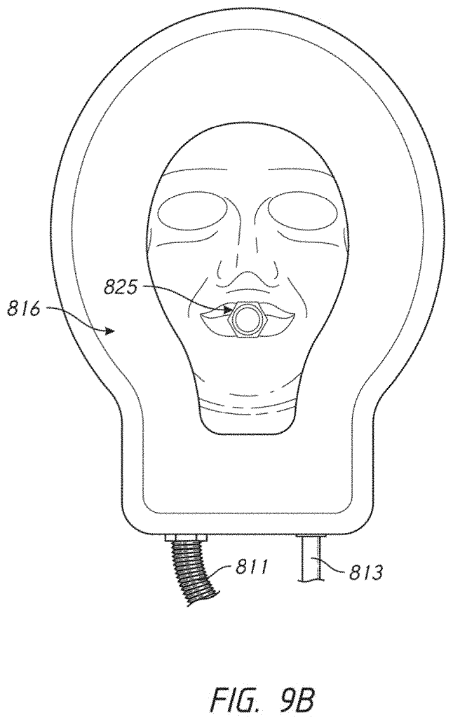

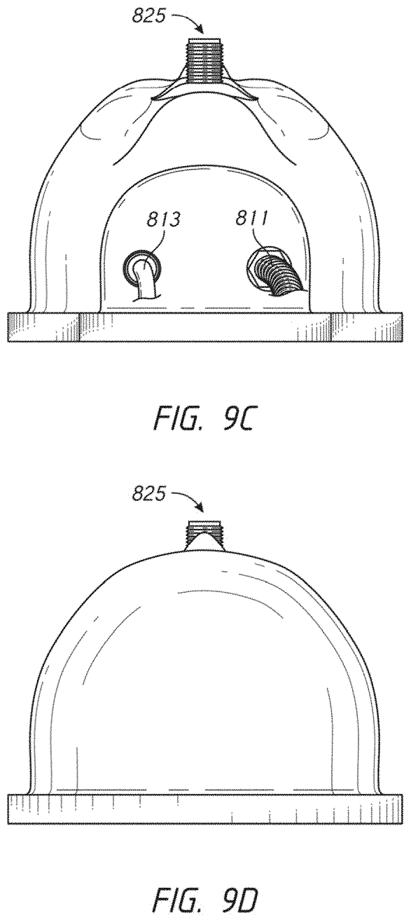

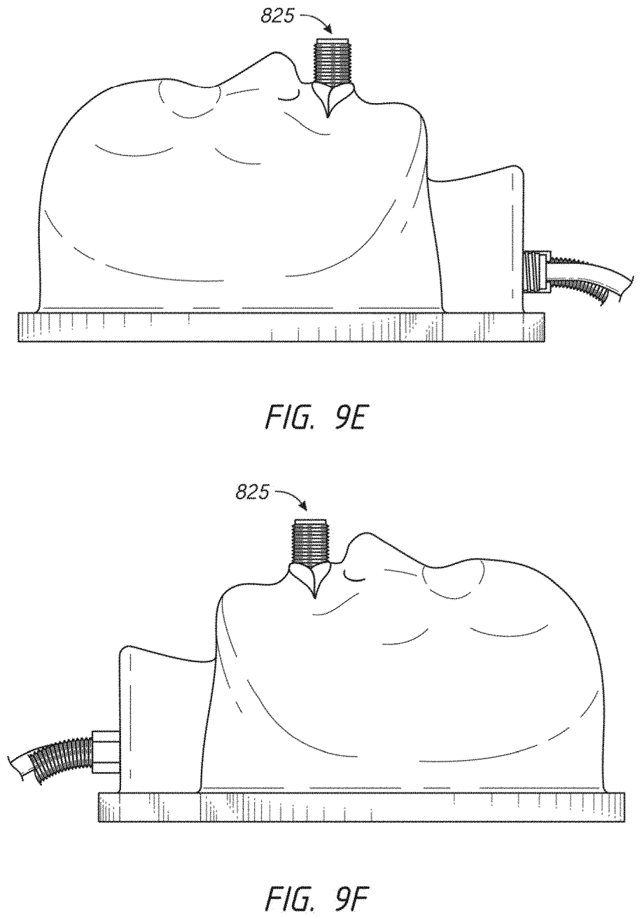

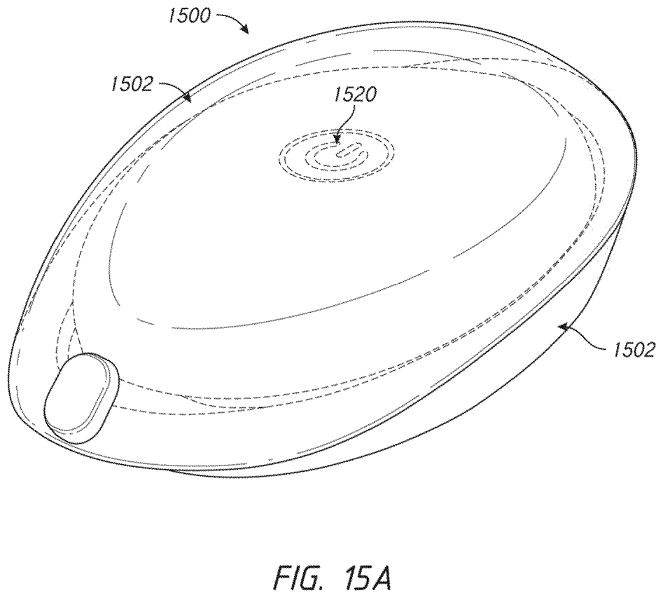

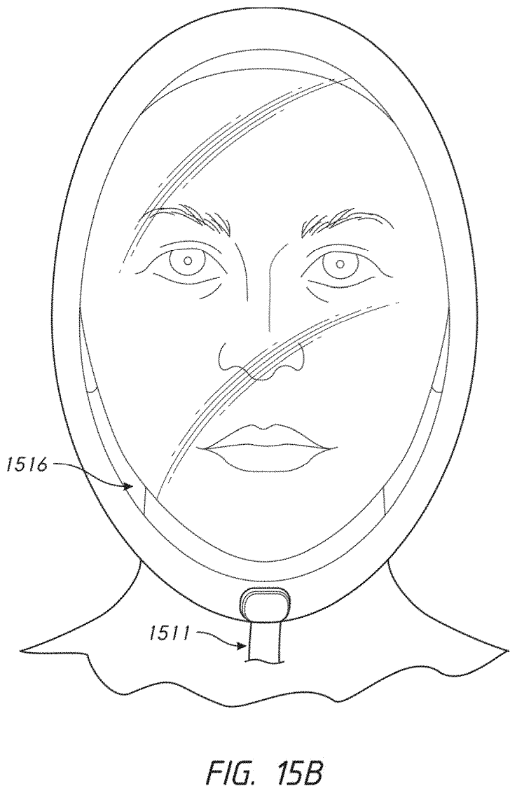

FIGS. 8A-8F illustrate various views of an example generator chamber 802 of a face priming device that can include any of the features of the face priming device 700 described above. The generator chamber 802 can include a power button 820 for activing an ozone generator located within the chamber 802. As shown in FIG. 8A, the power button 820 can be located on a side wall of the chamber 802. The power button can also be located at any other location on the chamber (for example, see the power button 1520 located on a top surface of the generator chamber 1502 in FIG. 15A). FIGS. 9A-9G illustrate various views of an example face mask 804 of a face priming device. The face mask 804 can be in fluid communication with the generation chamber 802 via tubing 811, 813. The flow of ozone can travel from the ozone generator in the chamber 802 via one of the tubing 811, 813 to the mask 804. Unused ozone can be recirculated from the mask 804 via the other one of the tubing 811, 813 back to the ozone generator. The face mask 804 can include a membrane 816 for sealing any air gap between the user's face and the mask 804. The face mask 804, including the membrane 816, the tubing 811, 813, and the generator chamber 802 can form a closed system for the flow of ozone. The face mask 804 can optionally include an opening with a tube segment 825 extending across the opening. When using the device, the user can use the mouth to breathe through the tube segment 825 so that ozone may not leak through the tube segment 825.

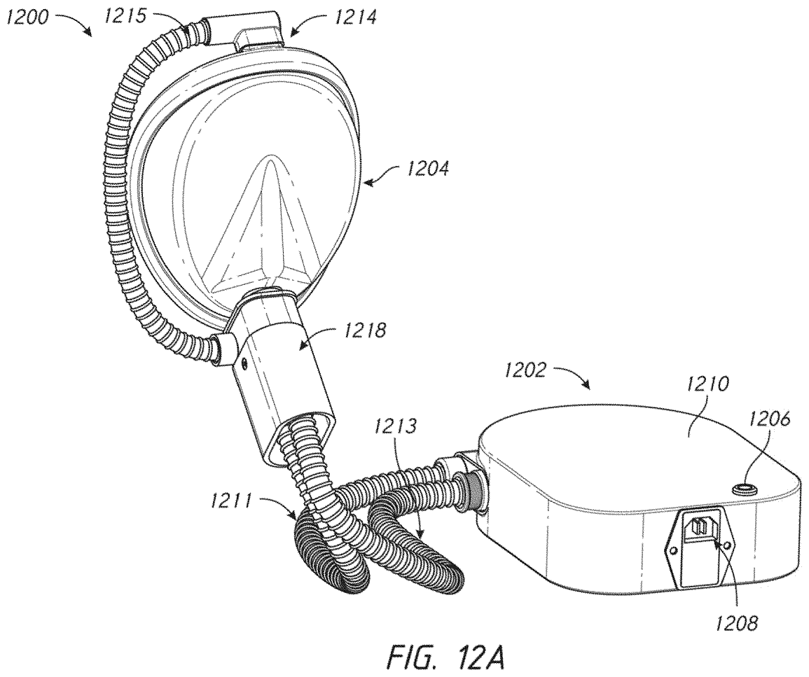

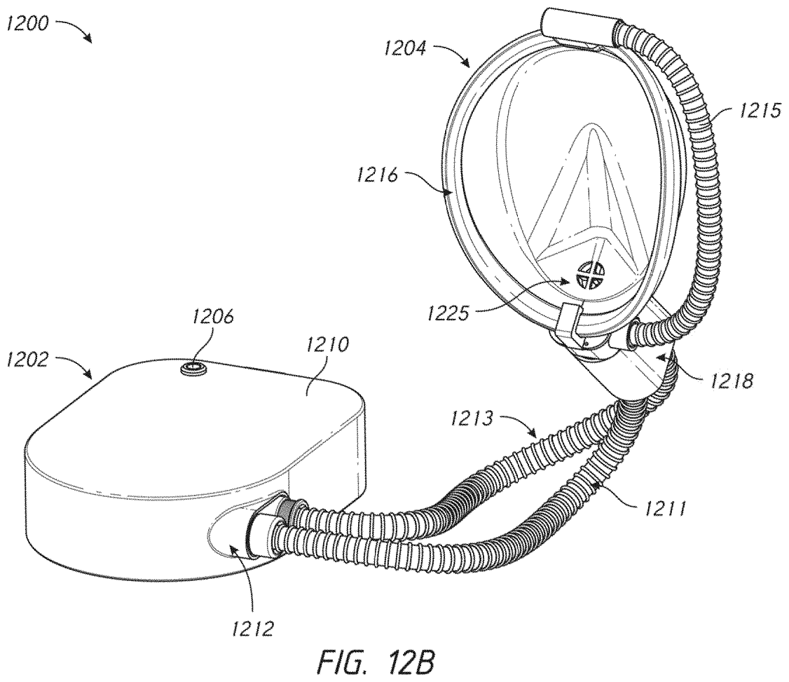

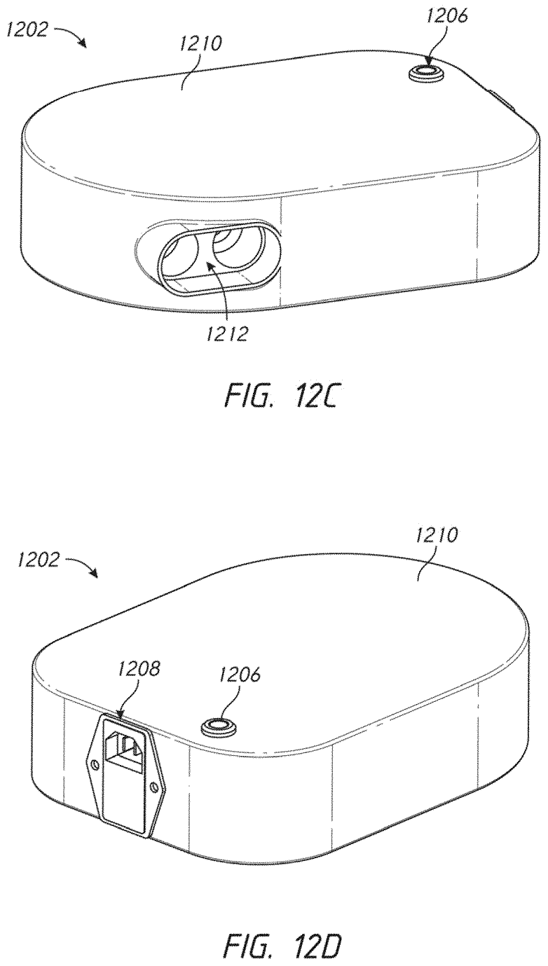

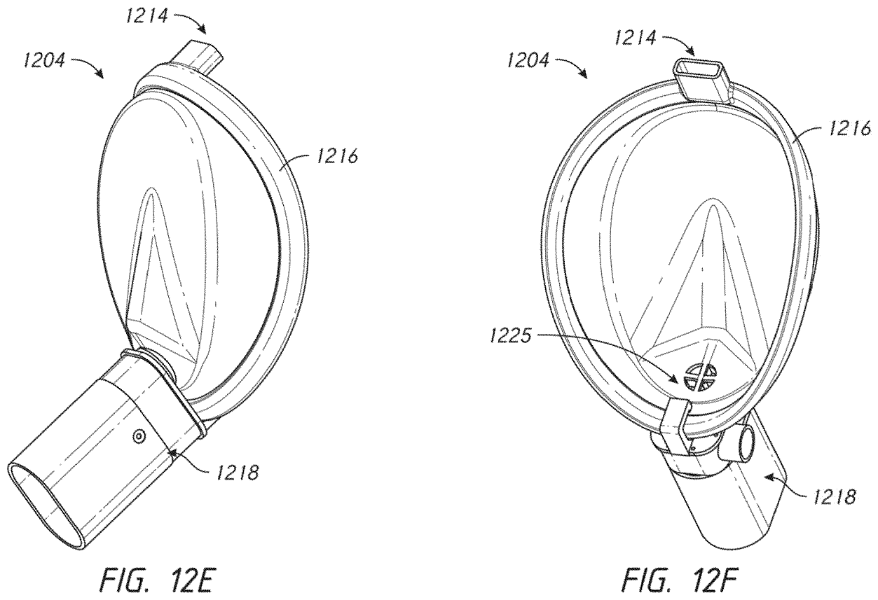

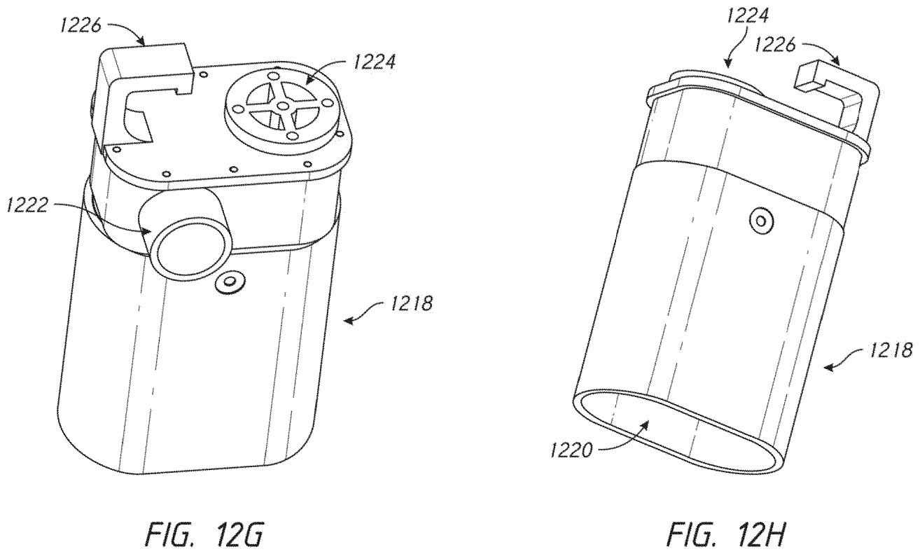

FIGS. 12A and 12B illustrate an example face priming device 1200. The face priming device 1200 can include any of the features of the face priming device examples described above. Any of the face priming device examples described above can also incorporate features of the face priming device 1200.

The device 1200 can include a face mask 1202 coupled to a generator chamber 1204 via tubing 1211, 1213. The face mask 1204 can be in fluid communication with the generation chamber 1202 via tubing 1211, 1213. The flow of ozone can travel from the ozone generator in the chamber 1202 via the tubing 1211 to the mask 1204. Unused ozone can be recirculated from the mask 1202 via the tubing 1213 back to the ozone generator.

As shown in FIGS. 12A-12D, the generator chamber 1202 can include a housing 1210. A power button 1206 can be located on a top surface or anywhere else on the housing 1210. A power plug 1208 can be located on a side wall or anywhere else on the housing 1210. The housing 1210 can also include tubing connecting ports 1212 on the side wall or anywhere else on the housing 1210. An ozone generator located within the housing 1210 can be in fluid communication with the ports 1212.

The face mask 1204 can include a membrane 1216 for sealing any air gap between the user's face and the mask 1204. The face mask 1204 can include an inlet 1214 for receiving a flow of ozone. Tubing 1215 can connect the inlet 1214 to the tubing 1211 so that the ozone generated by the ozone generator can flow into the space under the mask 1204 over the user's face. The face mask 1204 can have an outlet 1225. The tubing 1213 can connect between the outlet 1225 and the port 1212 of the chamber 1202 to return any unused ozone in the space under the mask 1204 back to the ozone generator. The face mask 1204, the tubing 1211, 1213, 1215, and the generator chamber 1202 can form a closed system for the flow of ozone. As shown in FIG. 12E and 12F, the inlet 1214 can be located on or near an upper end of the mask 1204 and the outlet 1225 can be located on or near a bottom end of the mask 1204. The locations of the inlet 1214 and the outlet 1225 can facilitate the flow of ozone over the surface of the user's face, for example, by gravity. The inlet 1214 and/or the outlet 1225 can also be located elsewhere on the face mask 1204.

The face priming device 1200 can optionally include an adapter 1218 such as shown in FIGS. 12G and 12H. The adapter 1218 can include a latch 1226 configured to couple the adapter 1218 to the face mask 1204, such as shown in FIGS. 12E and 12F. The adapter 1218 can include an opening 1220 for the tubing 1211, 1213 to be inserted into the adapter 1218. The adapter 1218 can include an incoming ozone outlet 1222, which can be connected to the tubing 1215 so that the flow of ozone from the tubing 1211 can flow from the generator chamber 1202 to the inlet 1214 of the mask 1204. The adapter 1218 can include a circulating ozone inlet 1224 which can be in fluid communication with the outlet 1225 of the face mask 1204 and the tubing 1213 so that the unused ozone in the mask 1204 can flow back to the ozone generator in the generator chamber 1202.

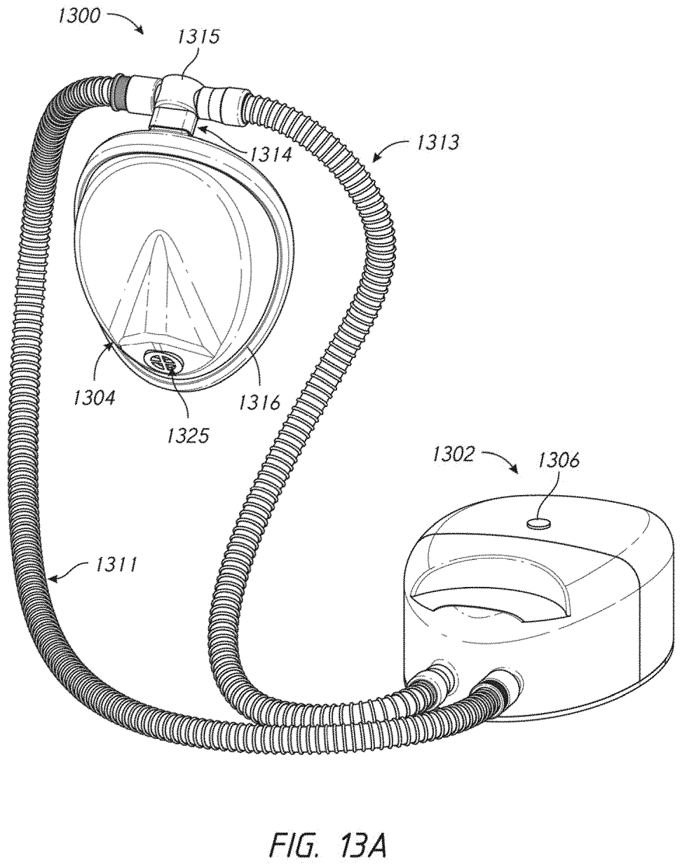

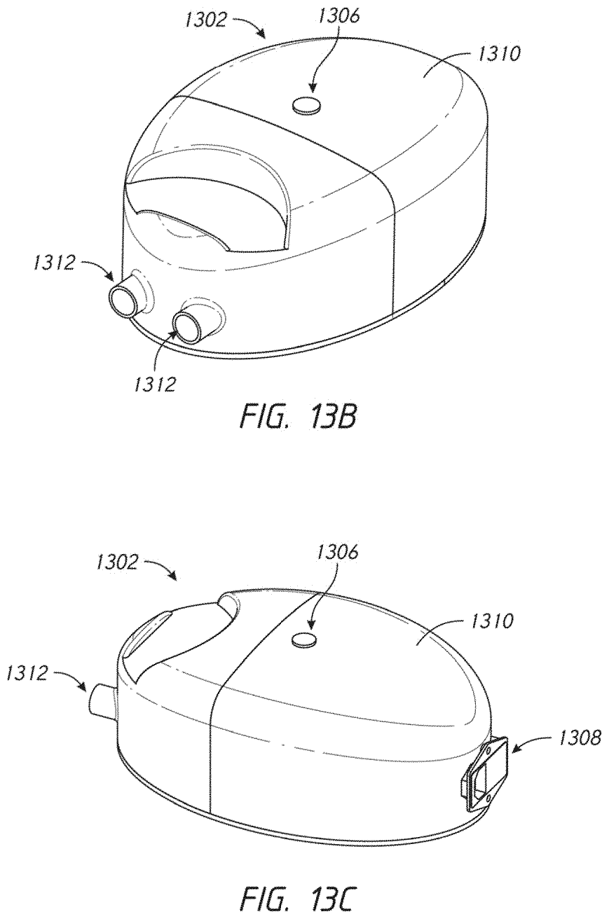

FIG. 13A illustrates an example face priming device 1300. The face priming device 1300 can include any of the features of the face priming device examples described above. Any of the face priming device examples described above can also incorporate features of the face priming device 1300.

The device 1300 can include a face mask 1302 coupled to a generator chamber 1304 via tubing 1311, 1313. The face mask 1304 can be in fluid communication with the generation chamber 1302 via tubing 1311, 1313. The flow of ozone can travel from the ozone generator in the chamber 1302 via the tubing 1311 to the mask 1304. Unused ozone can be recirculated from the mask 1302 via the tubing 1313 back to the ozone generator.

As shown in FIGS. 13B and 13C, the generator chamber 1302 can include a housing 1310. A power button 1306 can be located on a top surface or anywhere else on the housing 1310. A power plug 1308 can be located on a side wall or anywhere else on the housing 1310. The housing 1310 can also include tubing connecting ports 1312 on the side wall or anywhere else on the housing for coupling with the tubing 1311, 1313. An ozone generator located within the housing 1310 can be in fluid communication with the ports 1312.

The face mask 1304 can include an inlet/outlet 1314, which can be coupled to the tubing 1311, 1313 via a two-port connector 1315 (such as a wye-piece connector). Ozone can flow from the generator chamber 1302 via one of the tubing 1311, 1313 to the inlet/outlet 1314 of the face mask 1304. After having flown over the surface of the user's face, unused ozone can flow back to the generator chamber 1302 through the inlet/outlet 1314 via the other one of the tubing 1311, 1313. The face mask 1304 can include a membrane 1316 for sealing any air gap between the user's face and the mask 1304. The face mask 1304, including the membrane 1316, the tubing 1311, 1313, and the generator chamber 1302 can form a closed system for the flow of ozone.

The face mask 1304 can optionally include an opening 1325. When using the device, the user can use the mouth to breathe through the opening 1325 so that no ozone can leak through the opening 1325.

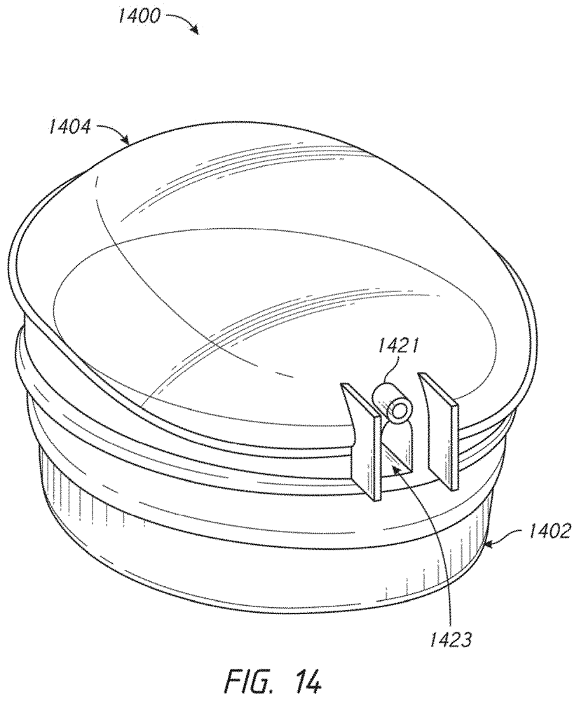

FIG. 14 illustrates a face priming device 1400 with tubing connecting the face mask 1404 and the generator chamber 1402 hidden to illustrate connection ports 1421, 1423 on the face mask 1404. The generator chamber 1402 can include corresponding connection ports similar to the connection ports 1312 shown in FIGS. 13B and 13C. The face priming device 1400 can have any of the features of the face priming devices described above. Any of the face priming device examples described above can also incorporate features of the face priming device 1400. As shown in FIG. 14, the face mask 1404 can be stacked onto the generator chamber 1402. The stacking can reduce the storage space for the face priming device 1400 and/or make the face priming device 1400 more compact and more portable.

FIG. 15A illustrates a face priming device 1500 with tubing connecting the face mask 1504 and the generator chamber 1502 hidden. The face priming device 1500 can have any of the features of the face priming devices described above. Any of the face priming device examples described above can also incorporate features of the face priming device 1500. As shown in FIG. 15A, the face mask 1504 can be stacked onto the generator chamber 1502. The stacking can reduce the storage space for the face priming device 1500 and/or making the face priming device 1500 more compact and more portable.

FIG. 15B illustrates the face mask 1504 being applied to a face of a user. The soft membrane 1516 can form a seal between the face mask 1504 and a perimeter of the user's face. Tubing 1211 can connect the face mask 1504 and the generator chamber 1502 for the flow of ozone. The face mask 1504 can have an optically transparent or see-through window such that the user can operate the device 1500, for example, by pressing the power button 1520 while wearing the mask 1504.

When the user removes the mask, which can be any of the masks disclosed herein, from the face, the user may detect an ozone odor. While this smell is detectible, cosmetics can be applied. The user can have about 2 minutes, about 3 minutes, about 5 minutes, or ranges between such values, to apply the first coat of cosmetics on the desired tissue before the ozone is oxidized by the air. Users can apply the face priming device to their faces more than once.

The face priming device examples disclosed herein can improve the condition of cosmetics on the user's face. For example, foundations applied after treatment can have a higher gloss and looking less greasy than foundations applied without treatment. The face priming device can also improve absorption of lotion into the skin. Cosmetics applied to the skin after having used the face priming device can have less flaking, caking, and/or clumping. One application of cosmetics, such as the foundation or otherwise, after having used the face priming device can last for over about 8 hours, about 10 hours, about 12 hours, about 14 hours, or ranges between such values, without re-application.

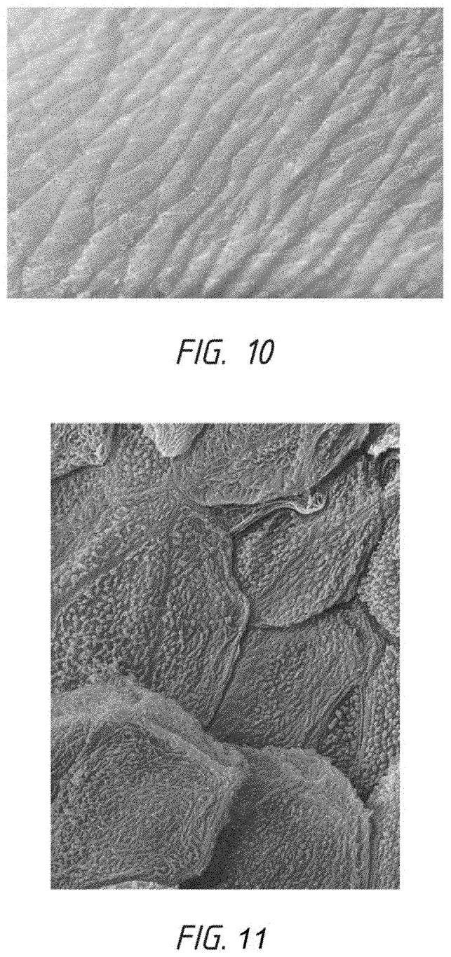

The improvement in cosmetics application using the face priming device can be due to similar surface energy reduction principles as seen in the improvement in nail polish application using the nail priming device disclosed herein. Better wetting, and/or more durable adhesion/longer adhesion time can improve the glossiness of the cosmetics on the user's face. As can be seen in FIGS. 10 and 11, the makeup of the skin can be considered similar to nail proteins in that the skin surface is full of areas that may not be fully wetted out due to micro air bubbles being trapped or where the cosmetics applied are not viscous enough to reach all the valleys in the skin. Voids in the skin surface can inhibit or prevent full contact between the skin and cosmetics. Increased surface energy can pull the cosmetics more tightly to the skin surface and cause the micro air bubble spaces to be filled by the cosmetics. Accordingly, application of the face priming device prior to the application of cosmetics can result in a more aesthetically pleasing (at least due to improved glossiness) and durable cosmetic application.

Further, the user's face may have a less “greasy” feeling after applying skin creams as the ointments can be absorbed better by the skin after application of the face priming device. Better absorption of the creams can also result in a softer feel and/or appearance to the skin compared to skin areas not pre-treated with ozone or another adhesion promoter using the face priming device.

In the face priming devices disclosed herein, ozone can be generated at high voltages and low currents. The voltage and/or currents required can vary depending on the rate of ozone generation. For example, an electrical power of about 90 W to about 100 W can be required to output ozone at about 10 g/hour. In some examples, the input voltage can be about DC 12V at about 8 A, about AC 110V at about 0.8 A, about AC 220V at about 0.4 A. In some examples, the output voltage can be about 3800 V at about 0.025 A, at a frequency of about 18 kHz to about 20 kHz. Less power can be used to generate ozone at a lower rate, such as 1 g/hour. An amount of gases, such as ozone, that can be generated in portable units may be limited by the battery size. The battery can be replaceable in the portable units. Additionally and/or alternatively, cartridges or canisters of ozone can be used to supplement the ozone generator in the portable units. Flexibility in the generation/storage of ozone can allow the face priming device (or the nail priming device) to have two sources of the ozone, to use the recirculation technology to reduce the total amount of ozone required per use and/or reduce the time taken for the residual ozone in the tubing, the mask, or the chamber to be oxidized, and to be appliance-based and portable. The recirculation in portable units can allow higher concentrations of surface energy enhancing gas and quicker application times than without recirculation.

Other devices can implement the ozone (or other adhesion promoter or surface energy enhancing gas) spray treatment onto other parts of a living organism. These devices can be portable or non-portable (for example, appliance-based). The devices can include a surface-energy enhancing gas (or adhesion promoter) generator, ways of transporting the gas using a fan or other pressure based transport method (blower), a sealed or semi-sealed chamber in which the tissue is exposed to the gas or aerosol for a limited amount of time (chamber), and a recirculation and/or filter system. The devices can be applied to, for example, the eye lashes, the eye lid area, a single nail, hair, eyebrow, or others. In some examples, the device can have a form factor suitable to be handheld and/or can be used to move over tissue areas. In some examples, the device can be a lipstick, a brush for mascara, eyebrow, eye shadow, and/or foundation, or any other cosmetic applicator having an integrated gas feed device (for example, through a lumen in the applicator). In some examples, the device can include a gas generator for use with cryogenic or other chambers prior to application of spray tan or other air brushed cosmetics. In some examples, a can of sun block lotion can include an ozone (or another adhesion promoter) containing pressurized cartridge that sprays the ozone to the skin prior to and/or simultaneous to the sun block spray being applied.

For appliance-based units, household appliances can be created for specific applications of the surface energy enhancing gas or vapor, such as the nail priming device described above. Appliances can also be similar to the face priming device, with masks or attachments that could be interchanged with an appliance-based generator unit. Each attachment can provide a circuit for the gas and/or a filter or a container for the gas. The attachments can include any of the other devices described above, for example, a wand (or similar appliance like a hair dryer) for use on hair (for example, for dyeing or for bikini waxing areas to improve the hold of hot wax (or similar) adhering to hairs to be removed). In some examples, the wand or other forms of attachments can be made of or include a rubber tube.

Appliance-based units can also include attached applicators, such as those described above, that have the makeup integrated into the applicator. For example, a foundation brush can allow the surface energy enhancing gas or adhesion promoter be applied through the handle or an integrated or otherwise connected tube so the gas is applied directly to the application area. A lipstick cartridge can be placed into a dispenser that applies the surface energy enhancing gas or adhesion promoter to the lips via a tube immediately prior to the lipstick is applied, and a recirculation nozzle or filter can be nearby to capture or reduce unused gas from getting into the environment. A mascara brush can include a similar gas tube system as well as any airbrush systems that apply spray cosmetics.

The portable system based on pressurized aerosols or battery gas generators can include similar portable dispensers as described for a household appliance unit. Mascara, lipstick, foundation, or any other cosmetic can be put into a specialized cartridge and dispensed with the primer (that is, the surface energy enhancing or adhesion promoter) gas with a continuous (or pulsed) and simultaneous gas treatment and cosmetic application rather than a pre-treatment followed by cosmetic application.

Other substances in addition to known adhesion promoters can improve surface energy of materials. Free electrons, ions, metastables, radicals, and UV generated in plasma regions can affect a surface with energies sufficient to break the molecular bonds on the surface of most substrates. This creates very reactive free radicals on a carbon-based tissue surface, which, in turn, can form cross-link, or in the presence of oxygen, react rapidly to form various chemical functional groups on the substrate surface and raise energy. Polar functional groups that can form and enhance bondability include carbonyl (C═O), carboxyl (HOOC), hydroperoxide (HOO—), and hydroxyl (HO—) groups. Any of these substances can be incorporated into a priming device.

In some examples, small amounts of reactive functional groups on a carbon-based tissue surface can improve surface characteristics and wettability. For example, concentrations of ozone generated at about 10 g/hour over about 15 seconds in a sealed circuit (approximately 0.08 g of ozone) can result in a sufficient amount of reactive groups to increase tissue wettability across a facial area of about 0.013 m² (about 20 in²), about 0.017 m² (about 27 in²), or about 0.020 m² (about 30 in²). In some examples, exposure above the concentration of about 10 g/hour, the exposure time of about 15 seconds, and/or the amount of about 0.62 mg, about 0.47 mg, or about 0.42 mg of ozone per cm2 (about 0.004 g, about 0.003 g, or about 0.0027 g of ozone per in²) over that facial area may not further noticeably improve the bonding of the cosmetics and the facial skin or nails.

Chemical primers exist for some cosmetics such as acid based and acid free fingernail primer. Chemicals such as these can be turned into aerosol form and used, or other alternatives (for example, lower cost alternatives) that perform the same or similar functions can also be used.

Other uses for improved surface energy in tissues include any applications that involve improving the performance adhesives, lotions, colorants, cosmetics, medicines or dyes, including, but not limited to:
improving the adherence of bandages for medical applications;
marking of animals for identification in herds or decorative coloring;
sticking bandaids or other medical adhesive bandages or tapes for home and/or hospital application;
adhering of dress up or movie masks improving retention of appearance props;
absorption of topically applied ointments for medical relief;
absorption of non-cosmetic creams including sports pain relief gels or burn relief lotions; and/or
absorption of sunblock creams, lotions, and/or sprays.

Although some example examples have been disclosed herein in detail, this has been done by way of example and for the purposes of illustration only. The aforementioned examples are not intended to be limiting with respect to the scope of the appended claims, which follow. It is contemplated by the inventors that various substitutions, alterations, and modifications may be made to the invention without departing from the spirit and scope of the invention as defined by the claims. For example, features described above in connection with one example can be used with a different example described herein and the combination still fall within the scope of the disclosure.

The drawings in this disclosure are intended to schematically illustrate certain examples and not to limit the disclosure. While the invention is susceptible to various modifications, and alternative forms, specific examples thereof have been shown in the drawings and are herein described in detail. It should be understood, however, that the invention is not to be limited to the particular forms or methods disclosed, but, to the contrary, the invention is to cover all modifications, equivalents, and alternatives falling within the spirit and scope of the various examples described and the appended claims. Any methods disclosed herein need not be performed in the order recited. The methods disclosed herein include certain actions taken by a practitioner; however, they can also include any third-party instruction of those actions, either expressly or by implication. For example, actions such as “applying cosmetics” include “instructing applying cosmetics.” The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers. For example, “about 1 g” includes “1 g.” Terms or phrases preceded by a term such as “substantially” include the recited term or phrase. For example, “substantially parallel” includes “parallel.” As another example, the terms “approximately,” “about,” “generally,” and “substantially” may refer to an amount that is within less than 10% of, within less than 5% of, within less than 1% of, within less than 0.1% of, and within less than 0.01% of the stated amount.

Conditional language, such as “can,” “could,” “might,” or “may,” unless specifically stated otherwise, or otherwise understood within the context as used, is generally intended to convey that certain examples include, while other examples do not include, certain features, elements, and/or steps. Thus, such conditional language is not generally intended to imply that features, elements, and/or steps are in any way required for one or more examples or that one or more examples necessarily include logic for deciding, with or without user input or prompting, whether these features, elements, and/or steps are included or are to be performed in any particular example.

1. A method of improving medical or cosmetics application, the method comprising:
placing a body part of a user in a substantially sealed chamber, wherein a surface energy enhancing fluid is delivered to a surface of the body part and increases surface energy of the surface of the body part by 10 mN/m to 20 mN/m for a duration;
removing the body part from the chamber; and
applying medicament or cosmetics to the surface of the body part within 3 minutes of removing the body part from the chamber.

2. The method of claim 1, wherein the body part comprises at least a portion of a face of the user.

3. The method of claim 1, wherein the body part comprises a nail, skin, or hair of the user.

4. The method of claim 1, wherein the surface energy enhancing fluid comprises ozone.

5. A method of increasing a surface energy of a portion of a body, the method comprising:
generating a surface energy enhancing fluid using a gas generator;
delivering the surface energy enhancing fluid to a surface of the portion of the body placed in a substantially sealed chamber, wherein the surface energy enhancing fluid is configured to increase the surface energy of the portion of the body by 10 mN/m to 20 mN/m; and
terminating the generating and/or delivering of the surface energy enhancing fluid after a duration.

6. The method of claim 5, wherein the duration is between 15 seconds and 35 seconds.

7. The method of claim 5, comprising recirculating remaining surface energy enhancing fluid within the substantially sealed system after the duration and/or absorbing remaining surface enhancing fluid using a filter.

8. (canceled)

9. The method of claim 5, wherein the surface energy enhancing fluid comprises ozone.

10. The method of claim 9, wherein the generating the surface energy enhancing fluid comprises applying an electrical current to oxygen in atmospheric air.

11. A method of increasing surface energy of a portion of a body, the method comprising:
delivering a surface energy enhancing fluid to the portion of the body, wherein the surface energy enhancing fluid is configured to increase the surface energy of the portion of the body; and
stopping delivering the surface energy enhancing fluid after a duration.

12. The method of claim 11, wherein the duration is between 15 seconds and 35 seconds.

13. (canceled)

14. (canceled)

15. The method of claim 11, further comprising circulating the surface energy enhancing fluid within a substantially sealed compartment and/or absorbing the surface energy enhancing fluid using a filter after the duration.

16. The method of claim 15, wherein the substantially sealed compartment comprises a sealing membrane configured to substantially seal the compartment upon applying the portion of the body to the compartment.

17. The method of claim 15, wherein the sealing membrane is configured to inhibit the surface energy enhancing fluid from exiting the compartment.

18. The method of claim 11, wherein a concentration of the surface energy enhancing fluid increases after every circulation of the surface energy enhancing fluid.

19. (canceled)

20. The method of claim 11, comprising outputting an alarm a second duration after the duration.

21. The method of claim 1, wherein the medicament or cosmetics comprises one or more of sprays, lotions, creams, ointment, or adhesives for medical or cosmetic purposes.

22. The method of claim 5, further comprising applying medicament or cosmetics to the surface of the body part.

23. The method of claim 22, wherein the medicament or cosmetics comprises one or more of sprays, gels, lotions, creams, ointment, or adhesives for medical or cosmetic purposes.

24. The method of claim 11, further comprising applying medicament or cosmetics to the surface of the body part.
